(19) United States
(12) Patent Application Publication
(10) Pub. No.: US 2013/0171184 A1
(43) Pub. Date: Jul. 4, 2013

(54) VACCINE AGAINST N. MENINGITIDIS

(71) Applicant: Mumtaz Virji, ST. Thomas, PA (US)

(72) Inventor: Mumtaz Virji, ST. Thomas, PA (US)

(73) Assignee: MACK TRUCKS INC., GREENSBORO, NC (US)

(21) Appl. No.: 13/820,261

(22) PCT Filed: Sep. 9, 2011
(86) PCT No.: PCT/EP11/65675

§ 371 Date: Mar. 1, 2013

(30) Foreign Application Priority Data
Sep. 10, 2010 (GB) 1015132.2

(51) Int. Cl. A61K 39/095

(52) U.S. Cl. CPC A61K 39/095
USPC 424/190.1; 424/249.1

(57) ABSTRACT

The present invention relates to immunogenic compositions and vaccines for the treatment and prevention of Neisserial disease. Immunogenic compositions of the invention comprise the Hsf (also called Msf) and Opc neisserial antigens. These antigens have been found to be the two mechanisms by which Neisseria meningitidis binds host vitronectin (in order to promote adhesion and/or to evade host complement mediated killing). Vaccines comprising both antigens can thus advantageously result in an immune response which is better able to kill neisserial strains and otherwise prevent vitronectin-related neisserial complement mediated killing resistance and/or neisserial adhesion events required for pathogenesis.

### TECHNICAL FIELD

The present invention relates to the field of Neisserial immunogenic compositions and vaccines, their manufacture and the use of such compositions in medicine. More particularly, it relates to vaccine compositions comprising a combination of Hsf and Opc antigens which has a quality of reducing complement mediated killing resistance of Neisserial pathogens.

### BACKGROUND

Neisserial strains of bacteria are the causative agents for a number of human pathologies, against which there is a need for effective vaccines to be developed. In particular Neisseria gonorrhoeae and Neisseria meningitidis cause pathologies which could be treated by vaccination.

Neisseria gonorrhoeae is the etiologic agent of gonorrhea, one of the most frequently reported sexually transmitted diseases in the world with an estimated annual incidence of 62 million cases (Gerbase et al 1998 Lancet 351; (Suppl 3) 2-4). The clinical manifestations of gonorrhea include inflammation of the mucus membranes of the urogenital tract, throat or rectum and neonatal eye infections. Ascending gonococcal infections in women can lead to infertility, ectopic pregnancy, chronic pelvic inflammatory disease and tubo-ovarian abscess formation. Septicemia, arthritis, endocarditis and menigitis are associated with complicated gonorrhea.

The high number of gonococcal strains with resistance to antibiotics contributes to increased morbidity and complications associated with gonorrhea. An attractive alternative to treatment of gonorrhea with antibiotics would be its prevention using vaccination. No vaccine currently exists for N. gonorrhoeae infections.

Neisseria meningitidis is an important pathogen, particularly in children and young adults. Septicemia and meningitis are the most life-threatening forms of invasive meningococcal disease (IMD). This disease has become a worldwide health problem because of its high morbidity and mortality.

Thirteen N. meningitidis serogroups have been identified based on antigenic differences in the capsular polysaccharides, the most common being A, B and C which are responsible for 90% of disease worldwide. Serogroup B is the most common cause of meningococcal disease in Europe, USA and several countries in Latin America.

Vaccines based on the capsular polysaccharide of serogroups A, C, W and Y have been developed and have been shown to control outbreaks of meningococcal disease (Peltola et al 1985 Pediatrics 76; 91-96). However serogroup B is poorly immunogenic and induces only a transient antibody response of a predominantly IgM isotype (Ala'Aldeen D and Cartwright K 1996, J. Infect. 33; 153-157). There is therefore no broadly effective vaccine currently available against the serogroup B meningococcus which is responsible for the majority of disease in most temperate countries. This is particularly problematic since the incidence of serotype B disease is increasing in Europe, Australia and America, mostly in children under 5. The development of a vaccine against serogroup B meningococcus presents particular difficulties because the polysaccharide capsule is poorly immunogenic owing to its immunologic similarity to human neural cell adhesion molecule. Strategies for vaccine production have therefore concentrated on the surface exposed structures of the meningococcal outer membrane but have been hampered by the marked variation in these antigens among strains.

Further developments have led to the introduction of vaccines made up of outer membrane vesicles which will contain a number of proteins that make up the normal content of the bacterial membrane. One of these is the VA-MENGOC-BC® Cuban vaccine against N. meningitidis serogroups B and C (Rodriguez et al 1999 Mem Inst. Oswaldo Cruz, Rio de Janeiro 94; 433-440). This vaccine was designed to combat an invasive meningococcal disease outbreak in Cuba which had not been eliminated by a vaccination programme using a capsular polysaccharide AC vaccine. The prevailing serogroups were B and C and the VA-MENGOC-BC® vaccine was successful at controlling the outbreak with an estimated vaccine efficiency of 83% against serogroup B strains of N. meningitidis (Sierra et al 1990 In Neisseria, Walter Gruyter, Berlin, M. Atchman et al (eds) p 129-134, Sierra et al 1991, NIPH Ann 14; 195-210). This vaccine was effective against a specific outbreak, however the immune response elicited would not protect against other strains of N. meningitidis.

Subsequent efficacy studies conducted in Latin America during epidemics caused by homologous and heterologous serogroup B meningococcal strains have shown some efficacy in older children and adults but its effectiveness was significantly lower in younger children who are at greatest risk of infection (Milagres et al 1994, Infect. Immun. 62; 4419-4424). It is questionable how effective such a vaccine would be in countries with multistrain endemic disease such as the UK. Studies of immunogenicity against heterologous strains have demonstrated only limited cross-reactive serum bactericidal activity, especially in infants (Tappero et al 1999, JAMA 281; 1520-1527).

A second outer membrane vesicle vaccine was developed in Norway using a serotype B isolate typical of those prevalent in Scandinavia (Fredriksen et al 1991, NIPH Ann, 14; 67-80). This vaccine was tested in clinical trials and found to have a protective efficacy after 29 months of 57% (Bjune et al 1991, Lancet, 338; 1093-1096).

However, the use of outer membrane vesicles in vaccines is associated with some problems. For instance, the OMV contain toxic lipopolysaccharides and they may contain immunodominant antigens which are either strain specific or are expressed variably. Several processes have been described which could be used to overcome some of the problems of outer membrane vesicle preparation vaccines. WO01/09350 describes processes that address some of these problems for instance by reducing toxicity and modifying the antigens present on the outer membrane vesicles.

There are diverse problems with the anti-meningococcal vaccines currently available. The protein based outer membrane vaccines tend to be specific and effective against only a few strains. The polysaccharide vaccines are also suboptimal since they tend to elicit poor and short immune responses, particularly against serogroup B (Lepow et al 1986; Peltola 1998, Pediatrics 76; 91-96).

Neisseria infections represent a considerable health care problem for which no vaccines are available in the case of N. gonorrhoeae or vaccines with limitations on their efficacy and ability to protect against heterologous strains are available in the case of N. meningitidis.

Furthermore it has been recognised that pathogenic neisseria can evade host complement mediated killing by coating itself with host vitronectin and factor H.

Clearly there is a need to develop superior vaccines against Neisserial infections that will improve on the efficacy of currently available vaccines and allow for protection against a wider range of strains. Furthermore, vaccines which have the capability of deactivating the bacterium's complement mediated killing resistance would be particularly useful.

### FIGURE LEGENDS

FIG. 1. Msf-Expressing Acapsulate H44/76 (GB) Derivatives Bind to Human Vn, Whether or not Opc is Expressed.

A. The activated form of Vn (aVn) was immobilised on ELISA plates and overlaid with different Nm phenotypes as shown. Meningococcal adhesion to aVn-coated plates was assessed by using anti-Nm antiserum and alkaline phosphatase-conjugated secondary antibodies. B. In a reverse experimental set up, immobilised bacteria were overlaid with aVn and the protein binding was examined using a polyclonal anti-Vn antiserum. Results are from one representative of duplicate experiments. C. Binding of Msf-expressing Nm to mouse, bovine and rabbit Vn compared with human Vn. Similar levels of the Msf-expressing bacteria bound to immobilised vitronectins, negligible binding was seen in its absence (both phenotypes were Opc-deficient variants).

FIG. 2. Msf- and Opc-Expressing Meningococci Bind to the Somatomedin B/Linker Region of aVn Via Distinct Mechanisms.

Sequences and substitutions of the biotinylated vitronectin peptides (amino acids 43-68) used: VA-26 is biotin-VTRGDVFTMPEDEYTVYDDGEEKNNA (SEQ ID NO: 15), VA-26S is biotin-VTRGDVFTMPEDEYsTVYsDDGEEKNNA (SEQ ID NO: 16), VA-26P is biotin-VTRGDVFTpMPEDEYTpVYDDGEEKNNA (SEQ ID NO: 17) [Ys=sulphated Y; Tp=phosphorylated T]. (A-C). ELISA plates coated with biotinylated Vn peptides VA-26 (A), VA-26S (B) or VA-26P(C) were used to assess the binding of various acapsulate H44/76 (GB series) phenotypes. Nm strain C751 isolates (Msf−) with and without Opc expression were included to highlight the specific requirement of the Opc protein (Sa E Cunha, Griffiths et al. 2010). In contrast to Opc that requires sulphated tyrosine (present only in VA-26S), Msf-expressing Nm bound equally well to all three peptides and significantly more than Opc-Msf-(A-C) or Msf-Opc-1− phenotypes (A and C); the latter binding only to VA-26S. D. The specificity of VA-26S binding was shown in an ELISA that incorporated biotinylated Vn peptide VA-26S as well as two unrelated peptides SY-30 and GS-22 of 30 and 22 residues (sequences shown below). E and F. Inhibition of Nm binding to immobilised aVn by VA-26S (E) or the mAb 8E6 (F). ELISA plates were coated with 2.5 μg/ml aVn and overlaid with bacteria preincubated with 25 μg/ml peptide for 30 min. For 8E6 inhibition, the mAb was added to aVn-containing wells at 10 μg/ml prior to the addition of bacteria. Nm binding was detected using anti-Nm antiserum and alkaline phosphatase-conjugated secondary antibody. SY-30: Bio-SGRGKGGKGL GKGGAKRHRK VLRDNIQGIY (SEQ ID NO:11)

### GS-22: Bio-GSNENQKTVK AESVPNMSFD QS (SEQ ID NO:12)

FIG. 3. The Vn-Peptide VA-26 is Recognised by Msf Proteins of Distinct Strains in Acapsulate and Fully Capsulate Meningococci.

Direct binding of several serogroup B Msf-overexpressing Nm is shown and their phenotypes clarified (see also Table 1). Strain G7-2 is a derivative of UK isolate MO1-240101 whereas G7-3 and G7-4 are strain H44/76-derived; G7-3 expresses the MO1-240101 Msf and G7-4 expresses the strain B16B6 Msf. Their binding to immobilised VA-26 was determined by ELISA. As Opc does not bind to this peptide, only the Msf-mediated binding can be seen. In addition, the levels of Opc expressed in the isolates were monitored and were found to be low or negligible.

FIG. 4. Direct Interactions of Msf with the Synthetic Vitronectin Peptide AR-36.

A. Synthetic biotinylated peptide AR-36 (spanning the Vn residues A360-R395 within the main heparin-binding region of human Vn; See Appendix 1 of the priority document) and a control peptide SY-30 (AR-36 and SY-30 sequences are shown below) were immobilised on extravidin coated plates and overlaid with the two acapsulate H44/76 (GB) phenotypes Msf+Opc+ (broken lines) and Msf+Opc− (solid lines). Bacterial binding was detected using anti-Nm antiserum. Significantly higher binding of AR36 over the control peptide could be seen and Msf expression alone seems to be sufficient for binding to the peptide as Msf+Opc+ and Msf+Opc− Nm bound to the same extent. The data are consistent with the previous observations that Opc can target HBD only by bridging via heparin (Sa E Cunha, Griffiths et al. 2010). A low level of binding of the Msf/Opc double mutant was also observed, which, when subtracted from the level of binding of Msf-expressers, a significant binding was still apparent (B).

### AR36: Biotin-A PRPSLAKKQR FRHRNRKGYR SQRGHSRGRN QNSRR (SEQ ID NO:13)

### SY-30: Biotin-SGRGKGGKGL GKGGAKRIIRK VLRDNIQGIY (SEQ ID NO:11)

FIG. 5. Purified Recombinant Msf Protein Binds to Activated Human Serum Vitronectin but not to Clusterin.

A. Purified freshly prepared recombinant Msf (active Msf, A) and denatured Msf proteins were immobilised on nitrocellulose and overlaid with 2.5 μg/ml of aVn or clusterin (Cln). Binding of the host proteins was detected with a polyclonal anti-Vn antibody or a mAb to clusterin. Purified recombinant Msf when correctly folded binds to human vitronectin; the binding is specific for activated Vn as it does not bind to Cln. Inset shows similar specific binding of purified recombinant Opc to Vn but not Cln. B. Msf-concentration dependent binding of aVn. Immobilised recombinant Msf was dotted onto nitrocellulose as shown and overlaid with 2.5 μg/ml of aVn and its binding detected as above.

FIG. 6. Msf-Expressing Meningococci Bind to Significant Levels of aVn from human serum; aVn-dependent serum resistance of Msf expressing isolates.

A. 5×10⁹ bacteria of the phenotypes shown were incubated in decomplemented 10% pooled human serum (PHS) for 1 h, harvested and washed before analysing for serum proteins bound by dot blotting (GB is acapsulate and G7-4 is capsulate H44/76 strain). Only Msf-expressing acapsulate and capsulate Nm bound to aVn analysed by mAb 8E6 binding. B and C. Relative serum resistance of acapsulate H44/76 isolates in PHS without added Vn (shown as −Vn in B) and increased serum resistance in the presence of aVn (+Vn in B) were assessed as described in Appendix 2 using 5% PHS. Comparison of serum resistance of the four acapsulate H44/76 phenotypes (B). Persistence of serum resistance by acapsulate H44/76 Msf-expressing Nm in 10% serum when serum is supplemented with aVn (C). D. Serum resistance of H44/76 fully capsulate phenotype over-expressing heterologous strain B16B6 Msf. The parental phenotype G7-4 expressed Opc but at low levels.

FIG. 7. Msf Increases Serum Resistance in aVn-Concentration Dependent Manner.

In a standard SBA, 1000 bacteria were exposed to PHS (5%) with or without added aVn as shown. Surviving bacteria were plated after 10 min for enumeration of surviving bacteria. Means and SE of triplicate estimations are shown.

FIG. 8. Msf and/or Opc-Expression Impart Considerable Survival Potential to Meningococci.

Relative survival of the four phenotypes (acapsulate H44/76 derivatives GB series) was assessed using a starting population in which non-expressers predominated. The SBA used standard conditions but contained 8×10³ cfu/100 μl at the start of the experiment (T=0 min). Total well contents were plated at the end of a 10 min exposure to 10% PHS or 10 μg/ml aVn supplemented PHS, cultured and enumerated. Data are means of two separate estimations, the range of values were within 15% of the mean values.

FIG. 9. Complement Factors C3 and C9 Deposition.

Meningococcal derivatives as shown were incubated in 10% PHS with and without added aVn for 8 min. The samples were chilled to terminate complement deposition and washed in cold PBS before dotting on to nitrocellulose for overlay with mAb to C9-neoantigen or polyclonal anti C3 antibody. (A) MAC deposition on the four derivatives of H44/76 acapsulate phenotypes in PHS without added aVn (−Vn) and aVn-supplemented PHS (+Vn). (B) Relative decrease in anti-C9 and anti-C3 antibody binding when Vn supplemented serum was compared with unsupplemented PHS.

FIG. 10. Capsulate Msf-Expressing MC58 and H44/76 Isolates Retard MAC Deposition by Binding to aVn.

A. Comparison of MAC deposition on acapsulate and capsulate phenotypes of H44/76 and MC58 strains was determined as outlined in legend to FIG. 9. The capsulate derivatives were exposed to serum for 15 min while acapsulate derivatives were exposed for 8 min. The roles of the Msf and Opc proteins are illustrated by presenting percent decrease in MAC binding of the Msf/Opc phenotypes compared to the double mutants. B and C. MAC deposition on capsulate H44/76 derivatives G7-4 and G7-4Δχ from decomplemented PHS (DS), 10% PHS or 10% PHS supplemented with 10 μg/ml aVn as assessed by the use of anti-C9 monoclonal and polyclonal antibodies. The phenotype G7-4 over-expresses strain B16B6 Msf and has low levels of Opc, whereas G7-4Δχ is a Msf++ Opc mutant.

FIG. 11. Synthetic Vn Peptide VA-26 Restores MAC Deposition in aVn-Supplemented Serum.

Using the method described in legend to FIG. 9, MAC deposition on the capsulate H44/76 derivative G7-4Δχ was examined in decomplemented serum (DS) or in 10% PHS without or with further supplements as shown. When added, activated Vn was present at 10 μg/ml and the peptides VA-26 (sequence shown in legend to FIG. 2) and MV-14 (sequence shown below) were present at 25 μg/ml. Mean values and SE of triplicate estimations of one of two representative experiments are shown.

MV-14 peptide sequence: Bio-MDFPVDTTEGPQRV (SEQ ID NO:14).

FIG. 12. Effect of Varying Opc Levels in Capsulate and Acapsulate Msf-Expressing Phenotypes

Various isolates of strains MC58 and H44/76 were incubated in PHS with or without aVn and bacterial survival was determined after 10 min (acapsulate Nm) and 30 min (capsulate Nm) exposure to PHS or PHS+aVn.

### DETAILED DESCRIPTION

The present inventors have found that the Neisserial Hsf antigen (also called Msf herein) binds host vitronectin, and that this antigen (in combination with Opc binding of vitronectin) can account for neisserial vitronectin-based complement mediated killing resistance. A vaccine is thus proposed which comprises these 2 major resistance factors (Hsf and Opc) which may induce host antibodies that will target these factors so as to deactivate these systems and promote complement mediated killing of the neisserial pathogen in the host. A further known neisserial resistance factor is Factor H binding protein (FHbp) which binds factor H and also contributes to complement mediated killing resistance. A general vaccine is thus further proposed further comprising FHbp in order to induce host antibodies that will target these factors in general so as to deactivate both vitronectin and factor H-based systems and promote complement mediated killing of the neisserial pathogen in the host.

Accordingly there is provided an immunogenic composition comprising neisserial Hsf and Opc antigens, and optionally neisserial FHbp antigen (which may be either or both of the 2 known immunological families A or B).

The Hsf antigen may be from N. meningitidis, in particular serogroup B. The Hsf antigen may a polypeptide comprising:
a) the amino acid sequence of SEQ ID NO: 2 (Hsf from Nmen strain H44/76),
b) an amino acid sequence which has at least 80, 85, 90, 95, or 99% sequence identity with SEQ ID NO: 2, or
c) an immunogenic fragment of at least 10, 15, 20, 25, 30 contiguous amino acids of SEQ ID NO:2.

The Hsf antigen of the invention may be capable of eliciting antibodies which can inhibit the binding of human vitronectin to a polypeptide of SEQ ID NO: 2 and/or can inhibit the binding of human vitronectin to a N. meningitidis bacterium expressing a polypeptide of SEQ ID NO: 2 within its outer membrane. The Hsf antigen may be present in the immunogenic composition at a dose sufficient to elicit antibodies in a human host which can inhibit the binding of human vitronectin to Hsf (for instance a polypeptide of SEQ ID NO: 2) and/or can inhibit the binding of human vitronectin to a N. meningitidis bacterium expressing Hsf (for instance a polypeptide of SEQ ID NO: 2) within its outer membrane. In this regard serum may be tested in in vitro tests by looking at inhibition of binding using serum [from human, or any suitable animal model source] generated by the compositions of the invention (for instance using techniques well known to a skilled person or similar to those described in the examples.

The Opc antigen may be from N. meningitidis, in particular serogroup B.

The Opc antigen may be a polypeptide comprising:
a) the amino acid sequence of SEQ ID NO: 4 (Opc from Nmen strain H44/76),
b) an amino acid sequence which has at least 80, 85, 90, 95, or 99% sequence identity with SEQ ID NO: 4, or
c) an immunogenic fragment of at least 10, 15, 20, 25, 30 contiguous amino acids of SEQ ID NO:4.

The Opc antigen of the invention may be capable of eliciting antibodies which can inhibit the binding of human vitronectin to a polypeptide of SEQ ID NO: 4 and/or can inhibit the binding of human vitronectin to a N. meningitidis bacterium expressing a polypeptide of SEQ ID NO: 4 within its outer membrane. The Opc antigen may be present in the immunogenic composition at a dose sufficient to elicit antibodies in a human host which can inhibit the binding of human vitronectin to Opc (for instance a polypeptide of SEQ ID NO: 4) and/or can inhibit the binding of human vitronectin to a N. meningitidis bacterium expressing Opc (for instance a polypeptide of SEQ ID NO: 4) within its outer membrane. In this regard serum may be tested in in vitro tests by looking at inhibition of binding using serum [from human, or any suitable animal model source] generated by the compositions of the invention (for instance using techniques well known to a skilled person or similar to those described in the examples.

The FHbp antigen may be from N. meningitidis, in particular serogroup B. It may be related to the known family A type or family B type, or the compositions of the invention may incorporate FHbp from both families. Family classification is generally described in Journal of Infectious Diseases 2009 vo. 200 No 3 pp 379-389.

The FHbp family A antigen of the invention may be a polypeptide comprising:
a) the amino acid sequence of SEQ ID NO: 5 (FHbp from Nmen strain 8047),
b) an amino acid sequence which has at least 70, 80, 85, 90, 95, or 99% sequence identity with SEQ ID NO: 5, or
c) an immunogenic fragment of at least 10, 15, 20, 25, 30 contiguous amino acids of SEQ ID NO:5.

The FHbp family B antigen of the invention may be a polypeptide comprising:
a) the amino acid sequence of SEQ ID NO: 6 (Fhbp from Nmen strain MC58),
b) an amino acid sequence which has at least 70, 80, 85, 90, 95, or 99% sequence identity with SEQ ID NO: 6, or
c) an immunogenic fragment of at least 10, 15, 20, 25, 30 contiguous amino acids of SEQ ID NO:6.

The FHbp family A antigen of the invention may be capable of eliciting antibodies which can inhibit the binding of human factor H to a polypeptide of SEQ ID NO: 5 and/or can inhibit the binding of human factor H to a N. meningitidis bacterium expressing a polypeptide of SEQ ID NO: 5 within its outer membrane. The FHbp family B antigen of the invention may be capable of eliciting antibodies which can inhibit the binding of factor H to a polypeptide of SEQ ID NO: 6 and/or can inhibit the binding of human factor H to a N. meningitidis bacterium expressing a polypeptide of SEQ ID NO: 6 within its outer membrane. The FHbp family A antigen of the invention may be present in the immunogenic composition at a dose sufficient to elicit antibodies in a human host which can inhibit the binding of human factor H to FHbp family A (for instance a polypeptide of SEQ ID NO: 5) and/or can inhibit the binding of human factor H to a N. meningitidis bacterium expressing FHbp family A (for instance a polypeptide of SEQ ID NO: 5) within its outer membrane. The FHbp family B antigen of the invention may be present in the immunogenic composition at a dose sufficient to elicit antibodies in a human host which can inhibit the binding of human factor H to FHbp family B (for instance a polypeptide of SEQ ID NO: 6) and/or can inhibit the binding of human factor H to a N. meningitidis bacterium expressing FHbp family B (for instance a polypeptide of SEQ ID NO: 6) within its outer membrane. In this regard serum may be tested in in vitro tests by looking at inhibition of binding using serum [from human, or any suitable animal model source] generated by the compositions of the invention (for instance using techniques well known to a skilled person or similar to those described in the examples).

The antigens of the invention may be present as purified subunit antigens or within the outer membrane of an outer membrane vesicle preparation.

The Hsf antigen within the outer membrane preparation may be made from a neisserial (in particular N. meningitidis) strain which expresses Hsf in the outer membrane at a level that is the same or greater than in strain H44/76. It may be upregulated (preferably recombinantly) within the outer membrane vesicle. The outer membrane preparation may be made from a neisserial (in particular N. meningitidis) strain which has more than one copy of the hsf gene that encodes the Hsf antigen, or which has the hsf gene under the control of a heterologous promoter (i.e. a promoter that does not normally drive expression of the gene). Preferably the heterologous promoter is a stronger promoter than the hsf gene promoter.

The Opc antigen within the outer membrane preparation may be made from a neisserial (in particular N. meningitidis) strain which expresses Opc in the outer membrane at a level which is the same or greater than in strain H44/76 or strain C751 (an Opc+ strain described in Sa E Cunha et al PLoS Pathogens Vol 6 2010 e1000911). Though the expression of Opc may be naturally downregulated in N. meningitidis, strains expressing Opc tend to be isolated from the nasopharynx. Furthermore Opc expressing strains/cells which may be used in the current invention may be readily found using known colony blotting techniques (for instance as described in the “Meningococcal strains” section of Methods and Materials of Rosenqvist et al. I&I vol 63 1995 pp 4642-4652).

The Opc antigen of the invention may be upregulated (preferably recombinantly) within the outer membrane vesicle. The outer membrane preparation of the invention may be made from a neisserial (in particular N. meningitidis) strain which has more than one copy of the opc gene that encodes the Opc antigen, or which has the opc gene under the control of a heterologous promoter. A heterologous promoter also has the potential advantage of being more stable than the wild-type promoter. Preferably the heterologous promoter is a stronger promoter than the opc gene promoter.

The outer membrane preparation(s) of the invention may be made from a neisserial (in particular N. meningitidis) strain(s) which expresses FHbp (Fhbp family A and/or FHbp family B) in the outer membrane at a level which is the same or greater than in strain 8047 (for FHbp family A) or MC58 (for FHbp family B). The FHbp (Fhbp family A and/or FHbp family B) antigen may be upregulated (preferably recombinantly) within the outer membrane vesicle(s). The outer membrane preparation(s) of the invention may be made from a neisserial (in particular N. meningitidis) strain(s) which has more than one copy of the fhbp (more than one fhbp family A and/or more than one fhbp family B) gene that encodes the FHbp (FHbp family A and/or FHbp family B) antigen, or which has the fhbp (fhbp family A and/or fhbp family B) gene under the control of a heterologous promoter—preferably a stronger promoter than the fhbp (fhbp family A and/or fhbp family B) gene promoter.

The outer membrane vesicle (OMV) preparations of the invention may be made by collecting OMV naturally sloughed off by the bacterium (NOMVs) or may be extracted by a detergent—typically deoxycholate (DOC). The concentration of detergent (e.g. DOC) used may be 0-0.5%, 0.1-0.4%, or 0.2-0.3%, in particular around or exactly 0, 0.1, 0.2, 0.3, 0.4 or 0.5% DOC. Higher levels will remove LOS from the bleb which can be reactogenic. Lower levels will retain larger amounts of lipoproteins such as FHbp.

OMV preparations are preferably made from strains which cannot make (or has been engineered not to make) capsular polysaccharide. Further, immunodominant variable antigens are preferably removed from the OMVs to improve the immune response—for instance PorA and/or FrpB. LOS within the OMV of the invention may be detoxified with by deleting functional expression of the msbB and/or htrB genes in the OMV production strain.

The immunogenic compositions of the invention may further comprise neisserial (e.g. N. meningitidis) NspA and/or PilC [WO01/09350, WO2004/014418] (either in a subunit or outer membrane vesicle composition). These proteins may also be involved in binding factors which can supplement the complement mediated killing resistance of neisseria.

The immunogenic composition of the invention may further comprise one or more bacterial capsular polysaccharides or oligosaccharides, in particular those derived from bacteria selected from the group consisting of: Neisseria meningitidis serogroup A, C, Y and W-135, Haemophilus influenzae b, Streptococcus pneumoniae, Group A Streptococci, Group B Streptococci, Staphylococcus aureus and Staphylococcus epidermidis. These may be conjugated to a protein carrier (a provide of T-cell epitopes).

Further methods of treatment or prevention of Neisserial (Neisseria meningitidis and/or Neisseria gonorrhoeae) disease in a human host (or associated medical uses) are provided by the present invention using the immunogenic compositions of the invention. Given that vitronectin and factor H may be used by neisserial pathogens to adhere to human endothelial cells and pass through into different compartments of the body (e.g. to enter the meninges), the immunogenic compositions of the invention may be used for use in prevention of such adhesion and/or for the prevention of N. meningitidis meningitis. The compositions of the invention may clearly also be used to reduce neisserial complement mediated killing resistance (through vitronectin and/or factor H binding mechanisms), and for improved complement mediated killing of neisserial pathogens.

Pharmaceutical compositions are also provided comprising antibodies specific for the Hsf and Opc antigens of the invention, which may be used for medical treatment or prevention or other uses as described herein.

Where a protein is specifically mentioned herein, it is preferably a reference to a native, full-length protein, and to its natural variants (i.e. to a native protein obtainable from a Neisserial, preferably meningococcal strain) but it may also encompass antigenic fragments thereof (particularly in the context of subunit vaccines). These are fragments (often specifically described herein) containing or comprising at least 10 amino acids, preferably 20 amino acids, more preferably 30 amino acids, more preferably 40 amino acids or most preferably 50 amino acids, taken contiguously from the amino acid sequence of the protein. In addition, antigenic fragments denotes fragments that are immunologically reactive with antibodies generated against the Neisserial full-length proteins or with antibodies generated by infection of a mammalian host with Neisseria. Antigenic fragments also includes fragments that when administered at an effective dose, elicit a protective immune response against Neisserial infection, more preferably it is protective against N. meningitidis and/or N. gonorrhoeae infection, most preferably it is protective against N. meningitidis serogroup B infection.

Also included in the invention are recombinant fusion proteins of Neisserial proteins of the invention, or fragments thereof. These may combine different Neisserial proteins or fragments thereof in the same polypeptide. Alternatively, the invention also includes individual fusion proteins of Neisserial proteins or fragments thereof, as a fusion protein with heterologous sequences such as a provider of T-cell epitopes or purification tags, for example: β-galactosidase, glutathione-S-transferase, green fluorescent proteins (GFP), epitope tags such as FLAG, myc tag, poly histidine, or viral surface proteins such as influenza virus haemagglutinin, tetanus toxoid, diphtheria toxoid, CRM197.

### Antigens of the Invention

NMB (and GNA) references refer to reference numbers to sequences which can be accessed from www.neisseria.org.

NspA is described in WO96/29412. PilC is described in Mol. Microbiol. 1997, 23; 879-892.

### Hsf

Hsf (WO99/31132) (NMB 0992) has a structure that is common to autotransporter proteins: a signal sequence, a passenger domain and an anchoring domain for attachment to the outer membrane. For example, Hsf from N. meningitidis strain H44/76 consists of a signal sequence made up of amino acids 1-51, a head region at the amino terminus of the mature protein (amino acids 52-479) that is surface exposed and contains variable regions (amino acids 52-106, 121-124, 191-210 and 230-234), a neck region (amino acids 480-509), a hydrophobic alpha-helix region (amino acids 518-529) and an anchoring domain in which four transmembrane strands span the outer membrane (amino acids 539-591).

Although full length Hsf may be used in immunogenic compositions of the invention, various Hsf truncates and deletions may also be advantageously used depending on the type of vaccine.

Where Hsf is used in a subunit vaccine, it is preferred that a portion of the soluble passenger domain is used; for instance the complete domain of amino acids 52 to 479, most preferably a conserved portion thereof, for instance the particularly advantageous sequence of amino acids 134 to 479. Preferred forms of Hsf may be truncated so as to delete variable regions of the protein disclosed in WO01/55182.

Preferred variants would include the deletion of one, two, three, four, or five variable regions as defined in WO01/55182. The above sequences and those described below, can be extended or truncated by up to 1, 3, 5, 7, 10 or 15 amino acids at either or both N or C termini.

Preferred fragments of Hsf therefore include the entire head region of Hsf, preferably containing amino acids 52-473. Additional preferred fragments of Hsf include surface exposed regions of the head including one or more of the following amino acid sequences; 52-62, 76-93, 116-134, 147-157, 157-175, 199-211, 230-252, 252-270, 284-306, 328-338, 362-391, 408-418, 430-440 and 469-479.

Where Hsf is present in an outer membrane vesicle preparation, it may be expressed as the full-length protein or preferably as an advantageous variant made up of a fusion of amino acids 1-51 and 134-591 (yielding a mature outer membrane protein of amino acid sequence 134 to the C-terminus). Preferred forms of Hsf may be truncated so as to delete variable regions of the protein disclosed in WO01/55182. Preferred variants would include the deletion of one, two, three, four, or five variable regions as defined in WO01/55182. Preferably the first and second variable regions are deleted. Preferred variants would delete residues from between amino acid sequence 52 through to 237 or 54 through to 237, more preferably deleting residues between amino acid 52 through to 133 or 55 through to 133. The mature protein would lack the signal peptide.

A preferred process of the invention is therefore a process for producing intra-bleb conjugated LOS (preferably meningococcal) comprising the steps of conjugating blebs in the presence of EDAC/NHS at a pH between pH 7.0 and pH 9.0 (preferably around pH 7.5), in 1-5% (preferably around 3%) sucrose, and optionally in conditions substantially devoid of NaCl (as described above), and isolating the conjugated blebs from the reaction mix.

The reaction may be followed on Western separation gels of the reaction mixture using anti-LOS (e.g. anti-L2 or anti-L3) mAbs to show the increase of LOS molecular weight for a greater proportion of the LOS in the blebs as reaction time goes on.

Yields of 99% blebs can be recovered using such techniques.

EDAC was found to be an excellent intra-bleb cross-linking agent in that it cross-linked LOS to OMP sufficiently for improved LOS T-dependent immunogenicity, but did not cross link it to such a high degree that problems such as poor filterability, aggregation and inter-bleb cross-linking occurred. The morphology of the blebs generated is similar to that of unconjugated blebs (by electron microscope). In addition, the above protocol avoided an overly high cross-linking to take place (which can decrease the immunogenicity of protective OMPs naturally present on the surface of the bleb e.g. TbpA or Hsf).

### Factor H Binding Protein (Known as FHbp, GNA1870, NMB 1870)

FHbp proteins are defined into two families, A and B, herein. In one aspect the family classification is disclosed in “Sequence Diversity of the Factor H Binding Protein Vaccine Candidate in Epidemiologically Relevant Strains of Serogroup B Neisseria meningitides. The Journal of infectious diseases 2009, vol. 200, n° 3, pp. 379-389”

In one aspect the family identity is assessed over region 136-254 of the mature sequence. In one aspect proteins in the same family have >80% identity based upon the sequence of fHbp starting from amino acid 136 of the mature protein to the C terminus. In one aspect proteins in different families have 50-75% identity based upon the sequence of fHbp starting from amino acid 136 of the mature protein to the C terminus.

In one aspect the family identity is assessed over region 113-135 of the mature sequence. In one aspect proteins in the same family have >69% identity based upon the region 113-135 of the mature amino acid sequence of fHbp.

In one aspect proteins in different families have <20% identity based upon the region 113-135 of the mature amino acid sequence of fHbp.

In one aspect Family A and B may be distinguished by the presence of one or more of the following amino acids:

| AA position | Family A | Family B |
|---|---|---|
| 98 | I | V |
| 102 | D/N | S |
| 106-107 | VV | LT |
| 111 | I | T |
| 140 | A | S |
| 142-143 | NQ | D/GK |
| 146 | No amino acid equivalent to | E/K |
| | position 146 in family B. | |
| 149 | K | m/r/s |
| 151 | E | T |
| 153 | H | R |
| 155 | K | T |
| 158 | S | G |
| 186 | T | S |
| 197-198 | EL | D/YI |
| 200 | A | P |
| 204 | S | R/H |
| 209 | L | S |
| 211-213 | DTR | SVL |
| 215-217 | GG/SE | NQA/D |
| 221 | T | S |
| 223 | H | S |
| 225-226 | AL | GI |
| 229-230 | DR | GK/Q |
| 234 | I | V |
| 239 | T | E |
| 242-246 | IG/REKV (SEQ ID NO: 9) | TA/VNGI (SEQ ID NO: 10) |
| 248 | E | H |
| 251 | I | L |
| 253 | G | A |

In one aspect family A and B comprises the following consensus sequence from region 113-135:

| (SEQ ID NO. 7) | |
|---|---|
| A | KINNPDK(I/T)DSLIN(Q/R)RSFLVSGLG |
| (SEQ ID NO. 8) | |
| B | Q(V/I/E)QD(S/P)E(D/H)S(G/R)(K/S)MVAKR(Q/R)F |
| | (R/K)IGDI(A/V) |

An example of a family B sequence (SEQ ID NO. 6) is strain MC58.

Other examples of family B species include strains H44/76, M982, M060240006, 03s-0408, and other examples will be well known to the skilled person.

An example of a family A sequence (SEQ ID NO. 5) is strain 8047:

Other examples of family A species include strains M1239, M981, M08_{—}240117, M97252153, and other examples will be well known to the skilled person.

### Opc

Opc is a transmembrane protein of the beta barrel family with five surface-exposed loops and which binds vitronectin (Sa E Cunha et al. 2010 PLoS Pathogens vol 6 e1000911; Prince et al. PNAS USA 2002 99:3417-3421). The protein is basic in nature and has a prominent surface loop 2. Immunogenic fragments of Opc that may be used in the compositions of the invention (particularly as a subunit component) include one or more of these 5 surface exposed loops—in particular loop 2 which is involved in binding vitronectin. Together the surface loops of Opc may form a positively charged crevice that may accommodate negatively charged molecules. Opc has been shown to beind to heparin-like molecules and to heparin suphate proteoglycans (HSPG) on human epithelial cells in vitro.

Vitronectin is one of the more abundant plama proteins circulating at 200-400 μg/mL in humans and makes up 0.2-0.5% of total plasma proteins.

### Immunogenic Compositions

An immunogenic composition is a composition comprising at least the Hsf and Opc antigens of the invention which is capable of generating an immune response when administered to a host. Preferably, such immunogenic preparations are capable of generating a protective immune response against Neisserial, preferably Neisseria meningitidis or Neisseria gonorrhoeae infection.

### Subunit Compositions

The immunogenic composition of the invention may be a subunit composition (or may be a mixture of a subunit composition with a Outer Membrane Vesicle (or bleb) preparation).

Subunit compositions are compositions in which the components have been isolated and purified to at least 50%, preferably at least 60%, 70%, 80%, 90% pure before mixing the components to form the antigenic composition.

Subunit compositions may be aqueous solutions of water soluble proteins. They may comprise detergent, preferably non-ionic, zwitterionic or ionic detergent in order to solubilise hydrophobic portions of the antigens. They may comprise lipids so that liposome structures could be formed, allowing presentation of antigens with a structure that spans a lipid membrane.

### Outer Membrane Vesicle Preparations

N. meningitidis serogroup B (menB) excretes outer membrane blebs in sufficient quantities to allow their manufacture on an industrial scale. An outer membrane vesicles may also be prepared via the process of detergent extraction of the bacterial cells (see for example EP 11243).

The immunogenic composition of the invention may also comprise an outer membrane vesicle preparation having one or more antigens of the invention which have been upregulated, preferably recombinantly. Such preparations can optionally also comprise either or both of LPS immunotype L2 and LPS immunotype L3.

The manufacture of bleb preparations from Neisserial strains may be achieved by any of the methods well known to a skilled person. Preferably the methods disclosed in EP 301992, U.S. Pat. No. 5,597,572, EP 11243 or U.S. Pat. No. 4,271,147, Frederikson et al. (NIPH Annals [1991], 14:67-80), Zollinger et al. (J. Clin. Invest. [1979], 63:836-848), Saunders et al. (Infect. Immun. [1999], 67:113-119), Drabick et al. (Vaccine [2000], 18:160-172) or WO 01/09350 (Example 8) are used. In general, OMVs are extracted with a detergent, preferably deoxycholate, and nucleic acids are optionally removed enzymatically. Purification is achieved by ultracentrifugation optionally followed by size exclusion chromatography. If 2 or more different blebs of the invention are included, they may be combined in a single container to form a multivalent preparation of the invention (although a preparation is also considered multivalent if the different blebs of the invention are separate compositions in separate containers which are administered at the same time [the same visit to a practitioner] to a host). OMV preparations are usually sterilised by filtration through a 0.2 μm filter, and are preferably stored in a sucrose solution (e.g. 3%) which is known to stabilise the bleb preparations.

Upregulation of proteins within outer membrane vesicle preparations may be achieved by insertion of an extra copy of a gene into the Neisserial strain from which the OMV preparation is derived. Alternatively, the promoter of a gene can be exchanged for a stronger promoter in the Neisserial strain from which the OMV preparation is derived. Such techniques are described in WO01/09350. Upregulation of a protein will lead to a higher level of protein being present in OMV compared to the level of protein present in OMV derived from unmodified N. meningitidis (for instance strain H44/76). Preferably the level will be 1.5, 2, 3, 4, 5, 7, 10 or 20 times higher.

Where LPS is intended to be an additional antigen in the OMV, a protocol using a low concentration of extracting detergent (for example deoxycholate or DOC) may preferably be used in the OMV preparation method so as to preserve high levels of bound LPS whilst removing particularly toxic, poorly bound LPS. The concentration of DOC used is preferably 0-0.5% DOC, 0.02-0.4% DOC, 0.04-0.3% DOC more preferably 0.06%-0.2% DOC or 0.08-0.15% DOC most preferably around or exactly 0.1% DOC. 0.5% DOC should be used for removing LPS.

“Stronger promoter sequence” refers to a regulatory control element that increases transcription for a gene encoding antigen of interest.

“Upregulating expression” refers to any means to enhance the expression of an antigen of interest, relative to that of the non-modified (i.e., naturally occurring) bleb. It is understood that the amount of ‘upregulation’ will vary depending on the particular antigen of interest but will not exceed an amount that will disrupt the membrane integrity of the bleb. Upregulation of an antigen refers to expression that is at least 10% higher than that of the non-modified bleb. Preferably it is at least 50% higher. More preferably it is at least 100% (2 fold) higher. Most preferably it is 3, 4, 5, 7, 10, 20 fold higher.

Again for the purpose of clarity, the terms ‘engineering a bacterial strain to produce less of said antigen’ or down regulation refers to any means to reduce the expression of an antigen (or the expression of a functional gene product) of interest, relative to that of the non-modified (i.e., naturally occurring bleb), preferably by deletion, such that expression is at least 10% lower than that of the non-modified bleb. Preferably it is at least 50% lower and most preferably completely absent. If the down regulated protein is an enzyme or a functional protein, the downregulation may be achieved by introducing one or more mutations resulting in a 10%, 20%, 50%, 80% or preferably a 100% reduction in enzymatic or functional activity.

The engineering steps required to modulate the expression of Neisserial proteins can be carried out in a variety of ways known to the skilled person. For instance, sequences (e.g. promoters or open reading frames) can be inserted, and promoters/genes can be disrupted by the technique of transposon insertion. For instance, for upregulating a gene's expression, a strong promoter could be inserted via a transposon up to 2 kb upstream of the gene's initiation codon (more preferably 200-600 bp upstream, most preferably approximately 400 bp upstream). Point mutation or deletion may also be used (particularly for down-regulating expression of a gene).

Such methods, however, may be quite unstable or uncertain, and therefore it is preferred that the engineering step is performed via a homologous recombination event. Preferably, the event takes place between a sequence (a recombinogenic region) of at least 30 nucleotides on the bacterial chromosome, and a sequence (a second recombinogenic region) of at least 30 nucleotides on a vector transformed within the strain. Preferably the regions are 40-1000 nucleotides, more preferably 100-800 nucleotides, most preferably 500 nucleotides). These recombinogenic regions should be sufficiently similar that they are capable of hybridising to one another under highly stringent conditions.

Methods used to carry out the genetic modification events herein described (such as the upregulation or downregulation of genes by recombination events and the introduction of further gene sequences into a Neisserial genome) are described in WO01/09350. Typical strong promoters that may be integrated in Neisseria are porA, porB, lgtF, Opa, p110, lst, and hpuAB. PorA and PorB are preferred as constitutive, strong promoters. It has been established that the PorB promoter activity is contained in a fragment corresponding to nucleotides −1 to −250 upstream of the initation codon of porB.

### Down Regulation/Removal of Variable and Non-Protective Immunodominant Antigens

Many surface antigens are variable among bacterial strains and as a consequence are protective only against a limited set of closely related strains. An aspect of this invention covers outer membrane vesicles of the invention in which the expression of other proteins is reduced, or, preferably, gene(s) encoding variable surface protein(s) are deleted. Such deletion results in a bacterial strain producing blebs which, when administered in a vaccine, have a stronger potential for cross-reactivity against various strains due to a higher influence exerted by conserved proteins (retained on the outer membranes) on the vaccinee's immune system. Examples of such variable antigens in Neisseria that may be downregulated in the bleb immunogenic compositions of the invention include PorA, PorB, Opa.

For example, these variable or non-protective genes may be down-regulated in expression, or terminally switched off. This has the advantage of concentrating the immune system on better antigens that are present in low amounts on the outer surface of blebs. By down-regulation it is also meant that surface exposed, variable immunodominant loops of the above outer membrane proteins may be altered or deleted in order to make the resulting outer membrane protein less immunodominant.

Methods for downregulation of expression are disclosed in WO01/09350. Preferred combinations of proteins to be downregulated in the bleb immunogenic compositions of the invention include PorA and OpA; PorA and FrpB; OpA and FrpB; PorA and OpA and FrpB.

Four different Opa genes are known to exist in the meningococcal genome (Aho et al. 1991 Mol. Microbiol. 5:1429-37), therefore where Opa is said to be downregulated in expression it is meant that preferably 1, 2, 3 or (preferably) all 4 genes present in meningococcus are so downregulated. Such downregulation may be performed genetically as described in WO 01/09350 or by seeking readily-found, natural, stable meningococcal strains that have no or low expression from the Opa loci. Such strains can be found using the technique described in Poolman et al (1985 J. Med. Micro. 19:203-209) where cells that are Opa⁻ have a different phenotype to cells expressing Opa which can be seen looking at the appearance of the cells on plates or under a microscope. Once found, the strain can be shown to be stably Opa⁻ by performing a Western blot on cell contents after a fermentation run to establish the lack of Opa.

Where upregulation of some antigens in the outer membrane vesicle is achieved by growth under iron limitation conditions, the variable protein FrpB (Microbiology 142; 3269-3274, (1996); J. Bacteriol. 181; 2895-2901 (1999)) will also be upregulated. The inventors have found that it is advantageous to down-regulate expression of FrpB under these circumstances by downregulating expression of the entire protein as described in WO01/09350 or by deleting variable region(s) of FrpB. This will ensure that the immune response elicited by the immunogenic composition is directed towards antigens that are present in a wide range of strains.

In an alternative embodiment of the invention, FrpB is downregulated in outer membrane vesicles which have been prepared from Neisseria strains not grown under iron limitation conditions.

### Detoxification of LPS

The blebs in the immunogenic compositions of the invention may be detoxified via methods for detoxification of LPS which are disclosed in WO01/09350. In particular methods for detoxification of LPS of the invention involve the downregulation/deletion of htrB and/or msbB enzymes which are disclosed in WO01/09350. The msbB and htrB genes of Neisseria are also called 1pxL1 and 1pxL2, respectively (WO 00/26384) and deletion mutations of these genes are characterised pnenoltypically by the msbB− mutant LOS losing one secondary acyl chain), and the htrB− mutatn LOS losing both secondary acyl chains. WO93/14155 and WO 95/03327 describe nontoxix peptide functional equivalents of polymycin B that may be used in compositions of the invention.

Such methods are preferably combined with methods of bleb extraction involving low levels of DOC, preferably 0-0.3% DOC, more preferably 0.05%-0.2% DOC, most preferably around or exactly 0.1% DOC.

### Cross-Reactive Polysaccharides

The isolation of bacterial outer-membrane blebs from encapsulated Gram-negative bacteria often results in the co-purification of capsular polysaccharide. In some cases, this “contaminant” material may prove useful since polysaccharide may enhance the immune response conferred by other bleb components. In other cases however, the presence of contaminating polysaccharide material in bacterial bleb preparations may prove detrimental to the use of the blebs in a vaccine. For instance, it has been shown at least in the case of N. meningitidis that the serogroup B capsular polysaccharide does not confer protective immunity and is susceptible to induce an adverse auto-immune response in humans. Consequently, outer membrane vesicles of the invention may be isolated from a bacterial strain for bleb production, which has been engineered such that it is free of capsular polysaccharide. The blebs will then be suitable for use in humans. A particularly preferred example of such a bleb preparation is one from N. meningitidis serogroup B devoid of capsular polysaccharide.

This may be achieved by using modified bleb production strains in which the genes necessary for capsular biosynthesis and/or export have been impaired. Inactivation of the gene coding for capsular polysaccharide biosynthesis or export can be achieved by mutating (point mutation, deletion or insertion) either the control region, the coding region or both (preferably using the homologous recombination techniques described above), or by any other way of decreasing the enzymatic function of such genes. Moreover, inactivation of capsular biosynthesis genes may also be achieved by antisense over-expression or transposon mutagenesis. A preferred method is the deletion of some or all of the Neisseria meningitidis cps genes required for polysaccharide biosynthesis and export. For this purpose, the replacement plasmid pMF121 (described in Frosh et al. 1990, Mol. Microbiol. 4:1215-1218) can be used to deliver a mutation deleting the cpsCAD (+galE) gene cluster.

The safety of antibodies raised to L3 or L2 LPS has been questioned, due to the presence of a structure similar to the lacto-N-neotetraose oligosaccharide group (Galβ1-4GlcNAcβ1-3Galβ1-4Glcβ1−) present in human glycosphingolipids. Even if a large number of people has been safely vaccinated with deoxycholate extracted vesicle vaccines containing residual amount of L3 LPS (G. Bjune et al, Lancet (1991), 338, 1093-1096; GVG. Sierra et al, NIPH ann (1991), 14, 195-210), the deletion of the terminal part of the LOS saccharidic is advantageous in preventing any cross-reaction with structures present at the surface of human tissues. In a preferred embodiment, inactivation of the lgtB gene results in an intermediate LPS structure in which the terminal galactose residue and the sialic acid are absent (the mutation leaves a 4GlcNAcβ1-3Galβ1-4Glcβ1− structure in L2 and L3 LOS). Such intermediates could be obtained in an L3 and an L2 LPS strain. An alternative and less preferred (short) version of the LPS can be obtained by turning off the lgtE gene. A further alternative and less preferred version of the LPS can be obtained by turning off the lgtA gene. If such an lgtA⁻ mutation is selected it is preferred to also turn off lgtC expression to prevent the non-immunogenic L1 immunotype being formed.

LgtB⁻ mutants are most preferred as the inventors have found that this is the optimal truncation for resolving the safety issue whilst still retaining an LPS protective oligosaccharide epitope that can still induce a bactericidal antibody response.

Therefore, immunogenic compositions of the invention further comprising L2 or L3 preparations (whether purified or in an isolated bleb) or meningococcal bleb preparations in general are advantageously derived from a Neisserial strain (preferably meningococcal) that has been genetic engineered to permanently downregulate the expression of functional gene product from the lgtB, lgtA, galE or lgtE gene, preferably by switching the gene off, most preferably by deleting all or part of the promoter and/or open-reading frame of the gene.

Where the above immunogenic compositions of the invention are derived from a meningococcus B strain, it is further preferred that the capsular polysaccharide (which also contains human-like saccharide structures) is also removed. Although many genes could be switched off to achieve this, the inventors have advantageously shown that it is preferred that the bleb production strain has been genetically engineered to permanently downregulate the expression of functional gene product from the siaD gene (i.e. downregulating α-2-8 polysialyltransferase activity), preferably by switching the gene off, most preferably by deleting all or part of the promoter and/or open-reading frame of the gene. Such an inactivation is described in WO 01/09350. The siaD (also known as synD) mutation is the most advantageous of many mutations that can result in removing the human-similar epitope from the capsular polysaccharide, because it one of the only mutations that has no effect on the biosynthesis of the protective epitopes of LOS, thus being advantageous in a process which aims at ultimately using LOS as a protective antigen, and has a minimal effect on the growth of the bacterium. A preferred aspect of the invention is therefore a bleb immunogenic preparation as described above which is derived from an lgtE⁻siaD⁻, an lgtA⁻siaD⁻ or, preferably, an lgtB⁻siaD⁻ meningococcus B mutant strain.

Although siaD⁻ mutation is preferable for the above reasons, other mutations which switch off meningococcus B capsular polysaccharide synthesis may be used. Thus bleb production strain can be genetically engineered to permanently downregulate the expression of functional gene product from one or more of the following genes: ctrA, ctrB, ctrC, ctrD, synA (equivalent to synX and siaA), synB (equivalent to siaB) or synC (equivalent to siaC) genes, preferably by switching the gene off, most preferably by deleting all or part of the promoter and/or open-reading frame of the gene. The lgtE⁻ mutation may be combined with one or more of these mutations. Preferably the lgtB⁻ mutation is combined with one or more of these mutations. A further aspect of the invention is therefore a bleb immunogenic preparation as described above which is derived from such a combined mutant strain of meningococcus B. The strain itself is a further aspect of the invention.

A Neisserial locus containing various lgt genes, including lgtB and lgtE, and its sequence is known in the art (see M. P. Jennings et al, Microbiology 1999, 145, 3013-3021 and references cited therein, and J. Exp. Med. 180:2181-2190 [1994]).

Where full-length (non-truncated) LOS is to be used in the final product, it is desirable for LOS not to be sialyated (as such LOS generates an immune response against the most dangerous, invasive meningococcal B strains which are also unsialylated). In such case using a capsule negative strain which has a deleted synA (equivalent to synX and siaA), synB (equivalent to siaB) or synC (equivalent to siaC) gene is advantageous, as such a mutation also renders menB LOS incapable of being sialylated.

In bleb preparations, particularly in preparations extracted with low DOC concentrations LPS may be used as an antigen in the immunogenic composition of the invention. It is however advantageous to downregulate/delete/inactivate enzymatic function of either the lgtE, lgtA (particularly in combination with lgtC), or, preferably, lgtB genes/gene products in order to remove human like lacto-N-neotetraose structures. The Neisserial locus (and sequence thereof) comprising the lgt genes for the biosynthesis of LPS oligosaccharide structure is known in the art (Jennings et al Microbiology 1999 145; 3013-3021 and references cited therein, and J. Exp. Med. 180:2181-2190 [1994]). Downregulation/deletion of lgtB (or functional gene product) is preferred since it leaves the LPS protective epitope intact.

In N. meningitidis serogroup B bleb preparations of the invention, the downregulation/deletion of both siaD and lgtB is preferred, (although a combination of lgtB⁻ with any of ctrA⁻, ctB⁻, ctrC⁻, ctrD⁻, synA⁻ (equivalent to synX⁻ and siaA⁻), synB⁻ (equivalent to siaB⁻) or synC⁻ (equivalent to siaC⁻) in a meningococcus B bleb production strain may also be used) leading to a bleb preparation with optimal safety and LPS protective epitope retention.

A further aspect of the invention is therefore a bleb immunogenic preparation as described above which is derived from such a combined mutant strain of meningococcus B. The strain itself is a further aspect of the invention.

Immunogenic composition of the invention may comprise at least, one, two, three, four or five different outer membrane vesicle preparations. Where two or more OMV preparations are included, at least one antigen of the invention is upregulated in each OMV. Such OMV preparations may be derived from Neisserial strains of the same species and serogroup or preferably from Neisserial strains of different class, serogroup, serotype, subserotype or immunotype. For example, an immunogenic composition may comprise one or more outer membrane vesicle preparation(s) which contains LPS of immunotype L2 and one or more outer membrane vesicle preparation which contains LPS of immunotype L3. L2 or L3 OMV preparations are preferably derived from a stable strain which has minimal phase variability in the LPS oligosaccharide synthesis gene locus.

Outer Membrane Vesicles Combined with Subunit Compositions

The immunogenic compositions of the invention may also comprise both a subunit composition and an outer membrane vesicle. There are several antigens that are particularly suitable for inclusion in a subunit composition due to their solubility. Examples of such proteins include the FHbp antigen of the invention or the Hsf passenger domain. The outer membrane vesicle preparation is ideal for carrying integral membrane proteins such as Hsf, NspA, PilC, Opc antigens of the invention. FHbp may also be carried by OMVs via the lipid tail of the lipoprotein.

### Immunogenic Compositions of the Invention

The immunogenic compositions of the invention may comprise antigens (proteins, LPS and polysaccharides) derived from Neisseria meningitidis serogroups A, B, C, Y, W-135 or Neisseria gonorrhoeae.

### Further Combinations

The immunogenic composition of the invention may further comprise bacterial capsular polysaccharides or oligosaccharides. The capsular polysaccharides or oligosaccharides may be derived from one or more of: Neisseria meningitidis serogroup A, C, Y, and/or W-135, Haemophilus influenzae b, Streptococcus pneumoniae, Group A Streptococci, Group B Streptococci, Staphylococcus aureus and Staphylococcus epidermidis.

A further aspect of the invention are vaccine combinations comprising the antigenic composition of the invention with other antigens which are advantageously used against certain disease states including those associated with viral or Gram positive bacteria.

In one preferred combination, the antigenic compositions of the invention are formulated with 1, 2, 3 or preferably all 4 of the following meningococcal capsular polysaccharides or oligosaccharides which may be plain or conjugated to a protein carrier: A, C, Y or W-135. Preferably the immunogenic compositions of the invention are formulated with A and C; or C; or C and Y. Such a vaccine containing proteins from N. meningitidis, preferably serogroup B may be advantageously used as a global meningococcus vaccine.

In a further preferred embodiment, the antigenic compositions of the invention, preferably formulated with 1, 2, 3 or all 4 of the plain or conjugated meningococcal capsular polysaccharides or oligosaccharides A, C, Y or W-135 (as described above), are formulated with a conjugated H. influenzae b capsular polysaccharide or oligosaccharides, and/or one or more plain or conjugated pneumococcal capsular polysaccharides or oligosaccharides. Optionally, the vaccine may also comprise one or more protein antigens that can protect a host against Streptococcus pneumoniae infection. Such a vaccine may be advantageously used as a global meningitis vaccine.

In a still further preferred embodiment, the immunogenic composition of the invention is formulated with capsular polysaccharides or oligosaccharides derived from one or more of Neisseria meningitidis, Haemophilus influenzae b, Streptococcus pneumoniae, Group A Streptococci, Group B Streptococci, Staphylococcus aureus or Staphylococcus epidermidis. The pneumococcal capsular polysaccharide antigens are preferably selected from serotypes 1, 2, 3, 4, 5, 6B, 7F, 8, 9N, 9V, 10A, 11A, 12F, 14, 15B, 17F, 18C, 19A, 19F, 20, 22F, 23F and 33F (most preferably from serotypes 1, 3, 4, 5, 6B, 7F, 9V, 14, 18C, 19F and 23F). A further preferred embodiment would contain the PRP capsular polysaccharides of Haemophilus influenzae. A further preferred embodiment would contain the Type 5, Type 8 or 336 capsular polysaccharides of Staphylococcus aureus. A further preferred embodiment would contain the Type I, Type II or Type III capsular polysaccharides of Staphylococcus epidermidis. A further preferred embodiment would contain the Type Ia, Type Ic, Type II or Type III capsular polysaccharides of Group B streptocoocus. A further preferred embodiment would contain the capsular polysaccharides of Group A streptococcus, preferably further comprising at least one M protein and more preferably multiple types of M protein.

Such capsular polysaccharides of the invention may be unconjugated or conjugated to a carrier protein such as tetatus toxoid, tetanus toxoid fragment C, diphtheria toxoid, CRM197, pneumolysin, Protein D (U.S. Pat. No. 6,342,224). The polysaccharide conjugate may be prepared by any known coupling technique. For example the polysaccharide can be coupled via a thioether linkage. This conjugation method relies on activation of the polysaccharide with 1-cyano-4-dimethylamino pyridinium tetrafluoroborate (CDAP) to form a cyanate ester. The activated polysaccharide may thus be coupled directly or via a spacer group to an amino group on the carrier protein. Preferably, the cyanate ester is coupled with hexane diamine and the amino-derivatised polysaccharide is conjugated to the carrier protein using heteroligation chemistry involving the formation of the thioether linkage. Such conjugates are described in PCT published application WO93/15760 Uniformed Services University.

The conjugates can also be prepared by direct reductive amination methods as described in U.S. Pat. No. 4,365,170 (Jennings) and U.S. Pat. No. 4,673,574 (Anderson). Other methods are described in EP-0-161-188, EP-208375 and EP-0-477508. A further method involves the coupling of a cyanogen bromide activated polysaccharide derivatised with adipic acid hydrazide (ADH) to the protein carrier by Carbodiimide condensation (Chu C. et al Infect. Immunity, 1983 245 256). Where oligosaccharides are included, it is preferred that they be conjugated.

Preferred pneumococcal proteins antigens are those pneumococcal proteins which are exposed on the outer surface of the pneumococcus (capable of being recognised by a host's immune system during at least part of the life cycle of the pneumococcus), or are proteins which are secreted or released by the pneumococcus. Most preferably, the protein is a toxin, adhesin, 2-component signal tranducer, or lipoprotein of Streptococcus pneumoniae, or fragments thereof. Particularly preferred proteins include, but are not limited to: pneumolysin (preferably detoxified by chemical treatment or mutation) [Mitchell et al. Nucleic Acids Res. 1990 Jul. 11; 18(13): 4010 “Comparison of pneumolysin genes and proteins from Streptococcus pneumoniae types 1 and 2.”, Mitchell et al. Biochim Biophys Acta 1989 Jan. 23; 1007(1): 67-72 “Expression of the pneumolysin gene in Escherichia coli: rapid purification and biological properties.”, WO 96/05859 (A. Cyanamid), WO 90/06951 (Paton et al), WO 99/03884 (NAVA)]; PspA and transmembrane deletion variants thereof (U.S. Pat. No. 5,804,193—Briles et al.); PspC and transmembrane deletion variants thereof (WO 97/09994—Briles et al); PsaA and transmembrane deletion variants thereof (Berry & Paton, Infect Immun 1996 December; 64(12):5255-62 “Sequence heterogeneity of PsaA, a 37-kilodalton putative adhesin essential for virulence of Streptococcus pneumoniae”); pneumococcal choline binding proteins and transmembrane deletion variants thereof; CbpA and transmembrane deletion variants thereof (WO 97/41151; WO 99/51266); Glyceraldehyde-3-phosphate—dehydrogenase (Infect. Immun. 1996 64:3544); HSP70 (WO 96/40928); PcpA (Sanchez-Beato et al. FEMS Microbiol Lett 1998, 164:207-14); M like protein, (EP 0837130) and adhesin 18627, (EP 0834568). Further preferred pneumococcal protein antigens are those disclosed in WO 98/18931, particularly those selected in WO 98/18930 and PCT/US99/30390.

The immunogenic composition/vaccine of the invention may also optionally comprise antigens providing protection against one or more of Diphtheria, tetanus and Bordetella pertussis infections. The pertussis component may be killed whole cell B. pertussis (Pw) or acellular pertussis (Pa) which contains at least one antigen (preferably 2 or all 3) from PT, FHA and 69 kDa pertactin. Typically, the antigens providing protection against Diphtheria and tetanus would be Diphtheria toxoid and tetanus toxoid. The toxoids may chemically inactivated toxins or toxins inactivated by the introduction of point mutations.

### Vaccine Formulations

A preferred embodiment of the invention is the formulation of the immunogenic composition of the invention in a vaccine which may also comprise a pharmaceutically acceptable excipient or carrier.

The manufacture of outer membrane vesicle preparations from any of the aforementioned modified strains may be achieved by any of the methods well known to a skilled person. Preferably the methods disclosed in EP 301992, U.S. Pat. No. 5,597,572, EP 11243 or U.S. Pat. No. 4,271,147 are used. Most preferably, the method described in WO 01/09350 is used.

Vaccine preparation is generally described in Vaccine Design (“The subunit and adjuvant approach” (eds Powell M. F. & Newman M. J.) (1995) Plenum Press New York).

The antigenic compositions of the present invention may be adjuvanted in the vaccine formulation of the invention. Suitable adjuvants include an aluminium salt such as aluminum hydroxide gel (alum) or aluminium phosphate, but may also be a salt of calcium (particularly calcium carbonate), iron or zinc, or may be an insoluble suspension of acylated tyrosine, or acylated sugars, cationically or anionically derivatised polysaccharides, or polyphosphazenes.

Suitable Th1 adjuvant systems that may be used include, Monophosphoryl lipid A, particularly 3-de-O-acylated monophosphoryl lipid A, and a combination of monophosphoryl lipid A, preferably 3-de-O-acylated monophosphoryl lipid A (3D-MPL) together with an aluminium salt (preferably aluminium phosphate). An enhanced system involves the combination of a monophosphoryl lipid A and a saponin derivative particularly the combination of QS21 and 3D-MPL as disclosed in WO 94/00153, or a less reactogenic composition where the QS21 is quenched with cholesterol as disclosed in WO96/33739. A particularly potent adjuvant formulation involving QS21 3D-MPL and tocopherol in an oil in water emulsion is described in WO95/17210 and is a preferred formulation.

The vaccine may comprise a saponin, more preferably QS21. It may also comprise an oil in water emulsion and tocopherol. Unmethylated CpG containing oligo nucleotides (WO 96/02555) are also preferential inducers of a TH1 response and are suitable for use in the present invention.

The vaccine preparation of the present invention may be used to protect or treat a mammal susceptible to infection, by means of administering said vaccine via systemic or mucosal route. These administrations may include injection via the intramuscular, intraperitoneal, intradermal or subcutaneous routes; or via mucosal administration to the oral/alimentary, respiratory, genitourinary tracts. Thus one aspect of the present invention is a method of immunizing a human host against a disease caused by infection of a gram-negative bacteria, which method comprises administering to the host an immunoprotective dose of the OMV preparation of the present invention.

The amount of antigen in each vaccine dose is selected as an amount which induces an immunoprotective response without significant, adverse side effects in typical vaccinees. Such amount will vary depending upon which specific immunogen is employed and how it is presented. Generally, it is expected that each dose will comprise 1-100 μg of protein antigen or OMV preparation, preferably 5-50 μg, and most typically in the range 5-25 μg.

An optimal amount for a particular vaccine can be ascertained by standard studies involving observation of appropriate immune responses in subjects. Following an initial vaccination, subjects may receive one or several booster immunisations adequately spaced.

The vaccines of the invention are preferably immunoprotective and non-toxic and suitable for paediatric or adolescent use.

By paediatric use it is meant use in infants less than 4 years old.

### Further Aspects of the Invention

Another aspect of the invention involves a method for treatment or prevention of Neisserial disease comprising administering a protective dose (or effective amount) of the vaccine of the invention to a host in need thereof. Neisseria meningitidis serogroups A, B, C, Y or W135 and/or Neisseria gonorrhoeae infection could be advantageously prevented or treated.

The invention also includes a use of the vaccine of the invention in the preparation of a medicament for treatment of prevention of Neisserial infection. Again Neisserial infection encompasses infection by Neisseria meningitidis serogroups A, B, C, Y, W-135 and/or Neisseria gonorrhoeae.

Another aspect of the invention is a genetically engineered Neisserial strain from which an outer membrane vesicle of the inventions (having at least two proteins of the invention recombinantly upregulated, as described above) may be derived. Such Neisserial strains may be Neisseria meningitidis or Neisseria gonorrhoeae.

Further aspects of the invention are methods of making the immunogenic composition or vaccine of the invention. These include a method comprising a step of mixing together at least two isolated antigens or proteins of the invention from Neisseria, which may be present in the form of blebs derived from the Neisserial strains of the invention, to make an immunogenic composition of the invention, and a method of making the vaccine of the invention comprising a step of combining the immunogenic composition of the invention with a pharmaceutically acceptable carrier.

Also included in the invention are methods of making the immunogenic composition of the invention comprising a step of isolating outer membrane vesicles of the invention from a Neisserial culture. Such a method may involve a further step of combining at least two outer membrane vesicle preparations, preferably wherein at least one outer membrane vesicle preparation contains LPS of immunotype L2 and at least one outer membrane vesicle preparation contains LPS of immunotype L3. The invention also includes such methods wherein the outer membrane vesicles are isolated by extracting with a concentration of DOC of 0-0.5%. DOC concentrations of 0.3%-0.5% are used to minimise LPS content. In OMV preparations where LPS is to be conserved as an antigen, DOC concentrations of 0-0.3%, preferably 0.05%-0.2%, most preferably of about 0.1% are used for extraction.

### Outer Membrane Preparations or OMPC or Ghost or Killed Whole Cell Vaccines

The inventors envisage that the above improvements to bleb preparations and vaccines can be easily extended to ghost or killed whole cell preparations and vaccines (with identical advantages). The modified Gram-negative strains of the invention from which the bleb preparations are made can also be used to made ghost and killed whole cell preparations. Methods of making ghost preparations (empty cells with intact envelopes) from Gram-negative strains are well known in the art (see for example WO 92/01791). Methods of killing whole cells to make inactivated cell preparations for use in vaccines are also well known. The terms ‘bleb [or OMV] preparations’ and ‘bleb [or OMV] vaccines’ as well as the processes described throughout this document are therefore applicable to the terms ‘ghost preparation’ and ‘ghost vaccine’, and ‘killed whole cell preparation’ and ‘killed whole cell vaccine’, respectively, for the purposes of this invention.

Similarly embodiments described herein relating to outer membrane vesicle [OMV] preparations or bleb preparations of the invention can equally be applied in all instances herein to any outer membrane preparation [outer membrane preparations of the invention]. Such outer membrane preparations are purified from host bacterial DNA and many other cell cytoplasmic components. Outer membrane preparations of the invention may thus be any known type of membrane preparation such as blebs, OMVs, ghosts, or outer membrane complex (OMPC).

Methods for making OMPC for use in the outer membrane preparations of the invention are well known in the art, for instance following the methods described in Zollinger et al. (J. Clin. Invest. [1979], 63:836-848, U.S. Pat. No. 4,451,446, U.S. Pat. No. 4,601,903, or U.S. Pat. No. 4,695,624.

Optionally the blebs [OMVs] of the invention have been prepared such that the LOS content of the blebs is 3-30, 5-25, 10-25, 15-22% LOS content as measured by silver staining after SDS-PAGE electrophoresis using purified LOS as a standard (see method of Tsai, J. Biol. Standardization (1986) 14:25-33). 20% LOS in meningococcal blebs can be achieved with a 0.1% low DOC extraction, which may remove loosely held LOS molecules, but conserve the majority of the antigen. 0.5% DOC extraction can result in around 5% LOS in the preparation.

Optionally the OMPC of the invention has been prepared such that the LOS content of the OMPC is under 3, 2, 1, 0.75, 0.5, or 0.25%. Preferably the LOS content is below 1%.

### Antibodies and Passive Immunisation

Another aspect of the invention is a method of preparing antibodies or an immune globulin for use in prevention or treatment of Neisserial infection comprising the steps of immunising a recipient with the vaccine of the invention and isolating immune globulin from the recipient. An immune globulin prepared by this method is a further aspect of the invention. A pharmaceutical composition comprising the immune globulin of the invention and a pharmaceutically acceptable carrier is a further aspect of the invention which could be used in the manufacture of a medicament for the treatment or prevention of Neisserial disease. A method for treatment or prevention of Neisserial infection comprising a step of administering to a patient an effective amount of the pharmaceutical preparation of the invention is a further aspect of the invention.

Inocula for polyclonal antibody production are typically prepared by dispersing the antigenic composition in a physiologically tolerable diluent such as saline or other adjuvants suitable for human use to form an aqueous composition. An immunostimulatory amount of inoculum is administered to a mammal and the inoculated mammal is then maintained for a time sufficient for the antigenic composition to induce protective antibodies.

The antibodies can be isolated to the extent desired by well known techniques such as affinity chromatography (Harlow and Lane Antibodies; a laboratory manual 1988).

Antibodies can include antiserum preparations from a variety of commonly used animals e.g. goats, primates, donkeys, swine, horses, guinea pigs, rats or man. The animals are bled and serum recovered.

An immune globulin produced in accordance with the present invention can include whole antibodies, antibody fragments or subfragments. Antibodies can be whole immunoglobulins of any class e.g. IgG, IgM, IgA, IgD or IgE, chimeric antibodies or hybrid antibodies with dual specificity to two or more antigens of the invention. They may also be fragments e.g. F(ab′)2, Fab′, Fab, Fv and the like including hybrid fragments. An immune globulin also includes natural, synthetic or genetically engineered proteins that act like an antibody by binding to specific antigens to form a complex.

A vaccine of the present invention can be administered to a recipient who then acts as a source of immune globulin, produced in response to challenge from the specific vaccine. A subject thus treated would donate plasma from which hyperimmune globulin would be obtained via conventional plasma fractionation methodology. The hyperimmune globulin would be administered to another subject in order to impart resistance against or treat Neisserial infection. Hyperimmune globulins of the invention are particularly useful for treatment or prevention of Neisserial disease in infants, immune compromised individuals or where treatment is required and there is no time for the individual to produce antibodies in response to vaccination. An additional aspect of the invention is a pharmaceutical composition comprising two of more monoclonal antibodies (or fragments thereof; preferably human or humanised) reactive against at least two constituents of the immunogenic composition of the invention, which could be used to treat or prevent infection by Gram negative bacteria, preferably Neisseria, more preferably Neisseria meningitidis or Neisseria gonorrhoeae and most preferably Neisseria meningitidis serogroup B.

Such pharmaceutical compositions comprise monoclonal antibodies that can be whole immunoglobulins of any class e.g. IgG, IgM, IgA, IgD or IgE, chimeric antibodies or hybrid antibodies with specificity to two or more antigens of the invention. They may also be fragments e.g. F(ab′)2, Fab′, Fab, Fv and the like including hybrid fragments.

Methods of making monoclonal antibodies are well known in the art and can include the fusion of splenocytes with myeloma cells (Kohler and Milstein 1975 Nature 256; 495; Antibodies—a laboratory manual Harlow and Lane 1988). Alternatively, monoclonal Fv fragments can be obtained by screening a suitable phage display library (Vaughan T J et al 1998 Nature Biotechnology 16; 535). Monoclonal antibodies may be humanised or part humanised by known methods.

All references or patent applications cited within this patent specification are incorporated by reference herein.

The terms “comprising”, “comprise” and “comprises” herein is intended by the inventors to be optionally substitutable with the terms “consisting of”, “consist of”, and “consists of”, respectively, in every instance.

### Method of Industrial Application of the Invention

The examples below are carried our using standard techniques, which are well known and routine to those of skill in the art, except where otherwise described in detail. The examples are illustrative, but do not limit the invention.

### Example 1

### Methods for Constructing Strains of Neisseria meningitidis Serogroup B Used in Outer Membrane Vesicle Preparations

WO01/09350 and WO2004/014418 provide detailed general methods for preparing outer membrane vesicles and manipulating the bacterial strains from which the outer membrane vesicles are derived. Methods are disclosed for downregulation of antigens or genes (such as PorA, lgtB, lgtE, frpB, msbB, htrB), removal of capsular polysaccharide, upregulation of antigens (such as Hsf, NspA).

### Example 2

### Neisseria Meningitidis Hsf (NhhA or Msf Herein) Interacts Directly with Human Vitronectin: The Interplay Between Meningococcal Hsf and Opc in Host Cell Adhesion and Serum Resistance

Recently, we have investigated two key functions of meningococcal outer membrane proteins, that of sequestering complement regulatory molecules which leads to serum resistance and that of binding to cellular receptors which enables them to cross human cellular barriers in the nasopharynx and at the vascular interface. In this study, we observed that meningococcal Hsf (Msf for brevity) imparts vitronectin (Vn) binding property to the bacterium. In addition, Opc and Msf utilise vitronectin in normal human serum (NHS) to achieve complementary as well as exclusive functions. Using synthetic Vn peptides, we have delineated the Msf binding regions of vitronectin. As with Opc, this interaction prolongs the survival of meningococci in NHS by inhibiting C9 polymerisation and terminal complement complex insertion into the Msf-containing bacterial membrane. Thus, when mixtures of phenotypes are exposed to NHS, those expressing Vn-binding proteins demonstrate a striking ability to resist killing over those lacking their expression. However, it appears that Msf and Opc are not equally efficient in cellular adhesion and invasion via binding to Vn. The data imply that vitronectin binding may be an important property for the survival of the pathogen in in vivo environments for which the bacterium has evolved a number of distinct adhesion mechanisms. The aim of this presentation is to describe the novel mechanisms of interactions at the molecular level and the functional characteristics of the outer membrane proteins as well as the host components that are manipulated to enable survival and barrier penetration.

### Introduction

Neisseria meningitidis (Nm, meningococcus) is a human specific bacterium. It has evolved a number of mechanisms of resistance to innate and acquired immune mechanisms of its host, which are key to its success as a coloniser and as a pathogen of a considerable potential. Human antibody and complement play important roles in controlling the spread of the pathogen, such that in most individuals, the bacterium remains confined to respiratory mucosa. However, in this environment also, the bacterium may encounter human antibody, complement factors and other serum proteins. One mechanism, by which meningococci may acquire resistance to antibody and complement-mediated killing, is by sequestration of complement control factors such as factor H, complement component C4 binding protein or vitronectin. The former two mechanisms have been described for N. meningitidis but the latter has remained unknown. We have recently found that Nm may utilize this pathway also for the control of complement function.

Vitronectin (Vn) is a multifunctional plasma glycoprotein with an important complement regulatory function. The functions of Vn overlap with clusterin (Cln, also known as SP-40, cytolysis inhibitor) and, like Vn, Cln may block insertion of the terminal complement complex or membrane attack complex (MAC, C5b-9) into cellular membrane to prevent cytolysis. They are found complexed with MAC (approx. 1 molecule of each per complex). Brandtzaeg and colleagues (Hogasen, Mollnes et al. 1994) examined the levels of vitronectin and clusterin during severe meningococcal disease and found their levels to be low in the acute phase of meningococcal disease which gradually normalized in surviving patients. In their studies, low levels of Vn, in particular, correlated with disease severity and activation of the coagulation, fibrinolysis and complement systems.

Vitronectin plays several important roles in coagulation pathways. For example, it inhibits fibrinolysis by binding to plasminogen activator inhibitor type 1 (PAI-1) and increasing its functional life time. Vn also plays a procoagulant role by its effect on antithrombin III activity. Many of the functions of vitronectin require activation or unfolding of vitronectin, which occurs on physiological ligand binding. The levels of activated vitronectin may be augmented during coagulation but activated vitronectin is rapidly consumed into complexes with its ligands and cleared. Thus, the overall pool of vitronectin is reduced during sepsis. However, such continued activation of vitronectin also means that activated or unfolded vitronectin is present at higher than normal levels during meningococcal sepsis. (Some properties of vitronectin are shown in Appendix 1 of the priority document).

Vitronectin also promotes cell attachment and binds to integrins via its RGD motif. Interestingly, native, folded Vn contains many cryptic sites or partially exposed sites including those involved in heparin-binding as well as two sulphated tyrosine residues (Y56 and Y59), which we have discovered to be important in Opc interactions; see below.

### Our Studies on Opc and Vitronectin:

Several years ago, we described that vitronectin (Vn) bound to Nm Opc protein and increased the capacity of Opc-expressing isolates for cellular adhesion and invasion (Virji, Makepeace et al. 1994; Virji, Makepeace et al. 1995). Our recent studies have investigated the mechanisms of Opc interactions with vitronectin and have employed a number of techniques, including adsorption of serum by a variety of meningococcal isolates (and analyses of adsorbed proteins), use of activated unfolded and native folded forms of vitronectin and of antibodies that inhibit binding to activated vitronectin, the use of synthetic tyrosine sulphated peptides of vitronectin as well as peptides containing the high-affinity heparin binding domain to assess the precise mechanisms of Opc targeting of vitronectin. We have shown that vitronectin can bind directly to Opc-expressing Nm via the sulphated tyrosine residues in its Connecting Region (CR, Appendix 1 of the priority document) (Sa E Cunha, Griffiths et al. 2010). In addition, Opc-expressing Nm can also bind to a lesser extent to the C-terminal heparin binding domain by a sandwich mechanism involving heparin.

Such binding of Opc to vitronectin enables bacteria to adhere and invade human endothelial cells including brain microvascular endothelial cells. In addition, vitronectin binding may enable Opc-expressing bacteria to become more resistant to complement mediated killing; this possibility had not been investigated previously for meningococci, prior to our recent studies (preliminary data presented at IPNC 2008; Griffiths and Virji to be submitted).

In addition to Opc-dependent acquisition of serum resistance via vitronectin targeting, we have made a novel discovery that the meningococcal protein Msf, initially called NhhA/Hsf (see below), also binds to vitronectin. This, as with Opc, contributes to increased serum resistance of meningococci. This is the first study to define the mechanism of serum resistance afforded by Msf and our observations are briefly described below.

### Studies on Meningococcal Msf

The gene designated nhhA (Neisseria hia homologue, (Peak, Srikhanta et al. 2000)) was identified in Nm strain MC58 as a homologue of the adhesin AIDA-I of Escherichia coli. It was subsequently found to be more closely related to the Hia and Hsf adhesins of Haemophilus influenzae (similarities: Hsf, 74%; Hia, 67%; AIDA-I, 47%). The gene was present on all (85/85) strains of Nm examined and by Western blot analysis, it was shown to be expressed in the majority of strains tested, although the levels of expression varied. It appears that nhhA occurs in meningococci but not gonococci. In addition, as it is most similar to Hsf, Haemophilus influenzae surface fibril, we have preferred to use the term Msf (meningococcal surface fibril). Several studies have shown that recombinant Msf is immunogenic and elicits bactericidal antibodies, and that convalescent sera contain anti-Msf antibodies.

In July 2008 within my laboratory, we began investigations on the potential functions of Msf. We studied the potential role of Msf in binding to human extracellular matrix and serum proteins. In initial studies, we assessed if Msf-expressing meningococci were more serum resistant, as this is one property that may be acquired through binding of serum proteins such as vitronectin. By using Msf-proficient and Msf-deficient Nm and in parallel analysing Opc isolates (as Opc itself has a prominent role in vitronectin binding and in serum resistance as outlined above), we could observe increased serum resistance of Msf+Opc+ over Msf+Opc− meningococci. Strain MC58 expresses detectable levels of Msf, although these do not reach the levels found in the H44/76 isolates engineered to over-express Msf. In addition, by using Msf mutants of strain MC58 we were able to show the roles of both Msf and Opc in increased resistance to the bactericidal property of normal human serum. These observations led us to examine the potential targeting of serum vitronectin by Msf. The following results are set out to demonstrate a) the binding of vitronectin by Msf-expressing acapsulate and capsulate isolates of meningococci, b) the regions/of vitronectin that may be targeted by Msf, c) serum resistance acquired by binding to the activated form of human Vn and d) that the mechanism of serum resistance via Vn-targeting involves inhibition of complement factor C9 polymerisation and of lytic pore formation. Thus vitronectin targeting by Msf or Opc can inhibit all the major complement pathway activities by controlling the final critical stages of complement action.

### Results

Msf is a vitronectin-binding protein, demonstration of the binding of Opc-deficient, Msf-expressing isolates to human vitronectin.

As Vn circulates in the blood mainly in a closed conformation as a nascent protein, we studied the potential of both the more abundant native Vn (nVn) and the less abundant unfolded, activated form of Vn (aVn) to bind to Msf-expressing Nm. It was apparent from initial studies that native Vn preparations which can spontaneously undergo conformational change on storage, bound at low levels to Msf-expressing as well as Opc-expressing Nm. (Notably, the levels of activated vitronectin can be monitored by the use of conformation-dependent monoclonal antibody (mAb) 8E6 which binds only to activated Vn.) As we have previously established the absolute requirement for the activated form of Vn for Opc interactions (Sa E Cunha, Griffiths et al. 2010), the observations suggested that a similar mechanism to Opc binding may also be used by Msf-expressing Nm. In these studies we could establish that the nVn preparation we used contained approximately 25% unfolded mAb 8E6 binding component. Concurrently, the binding of Opc or Msf expressing bacteria to this nVn preparation was ˜30% of that found with the fully activated preparation of Vn. Thus it can be concluded that Msf preferentially binds to the conformationally activated form of Vn.

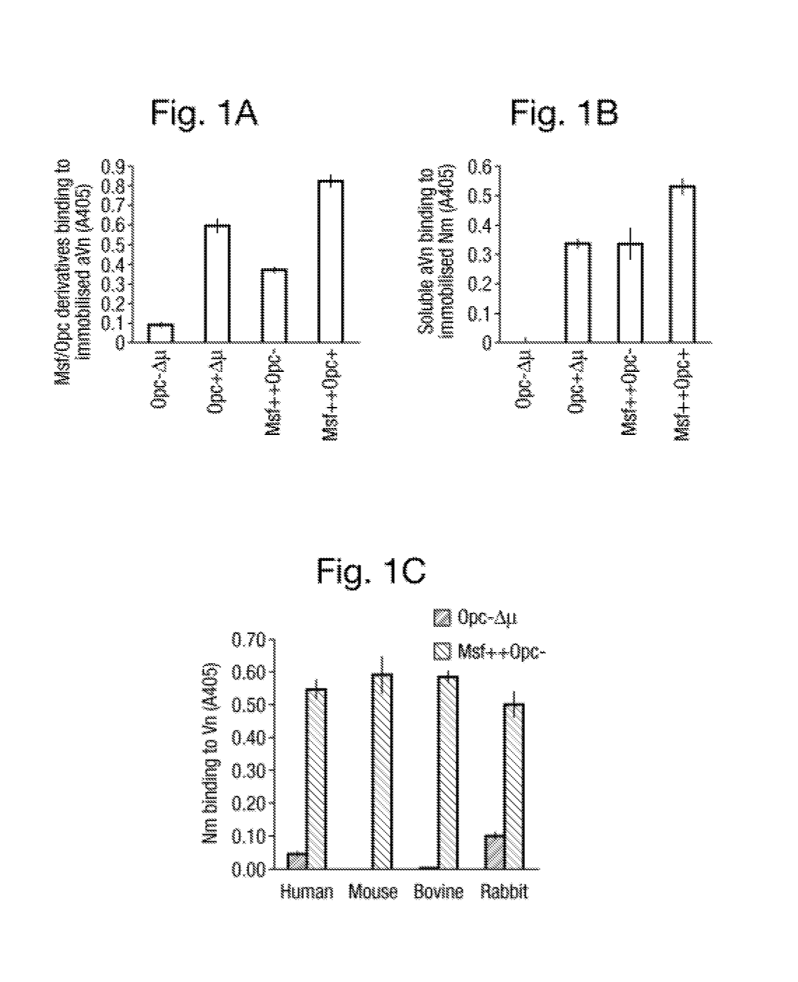

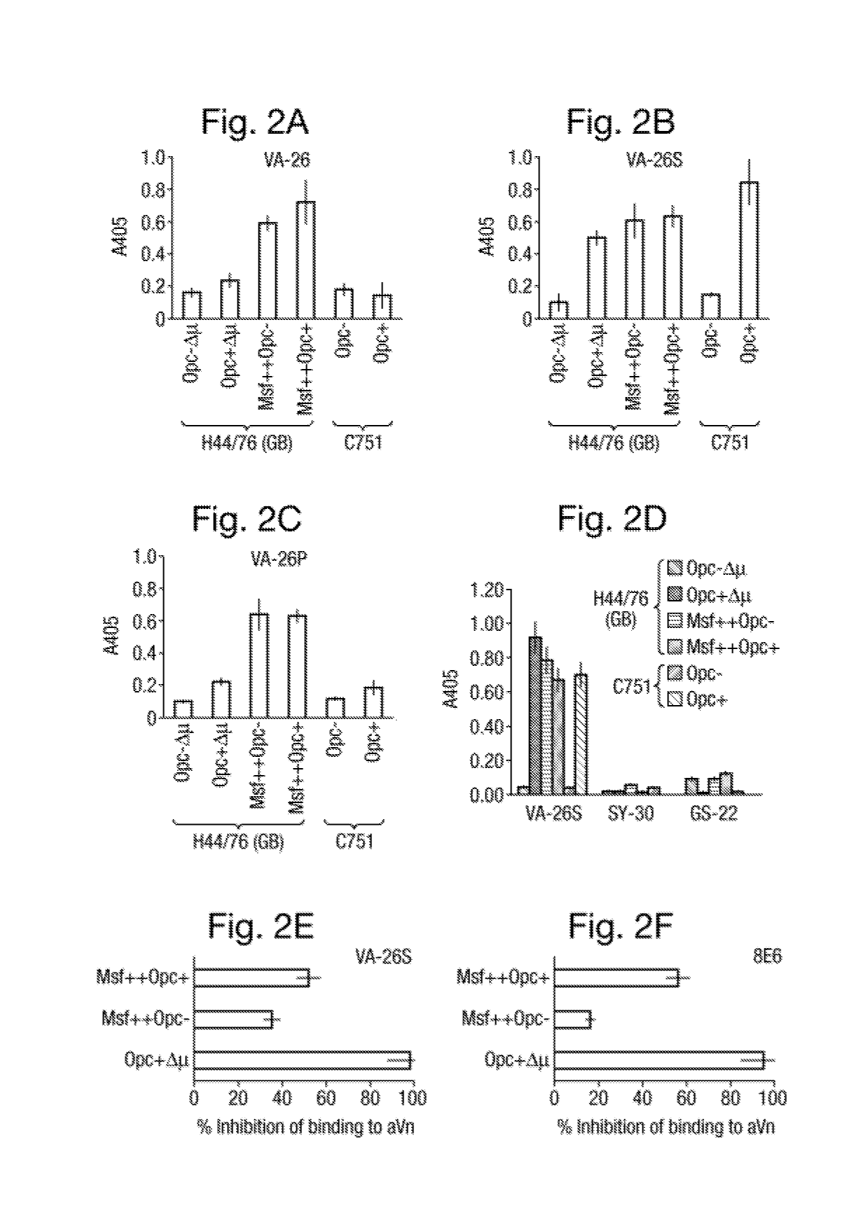

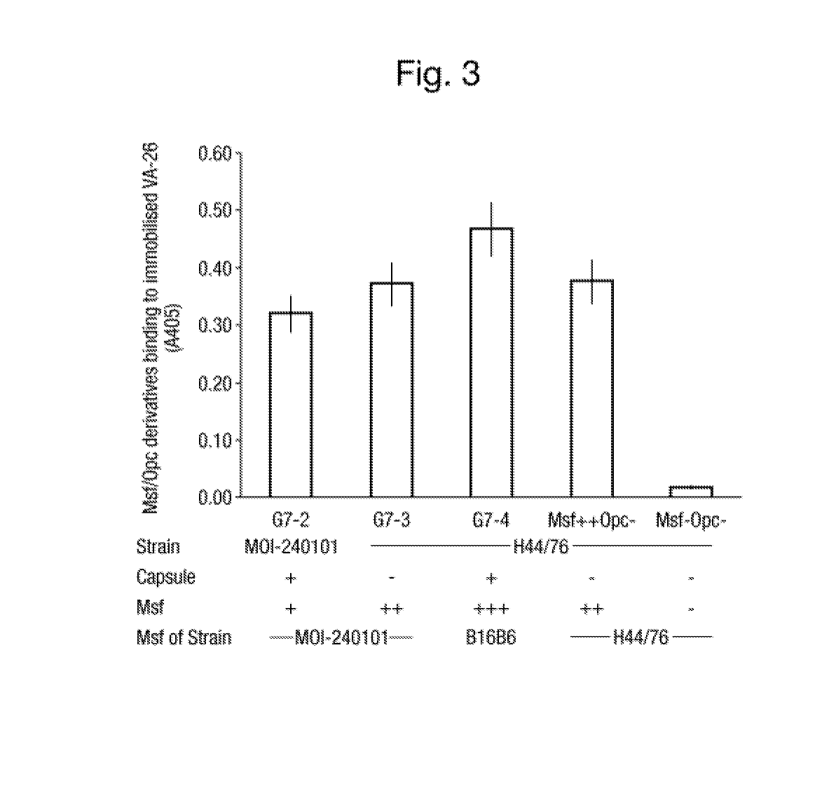

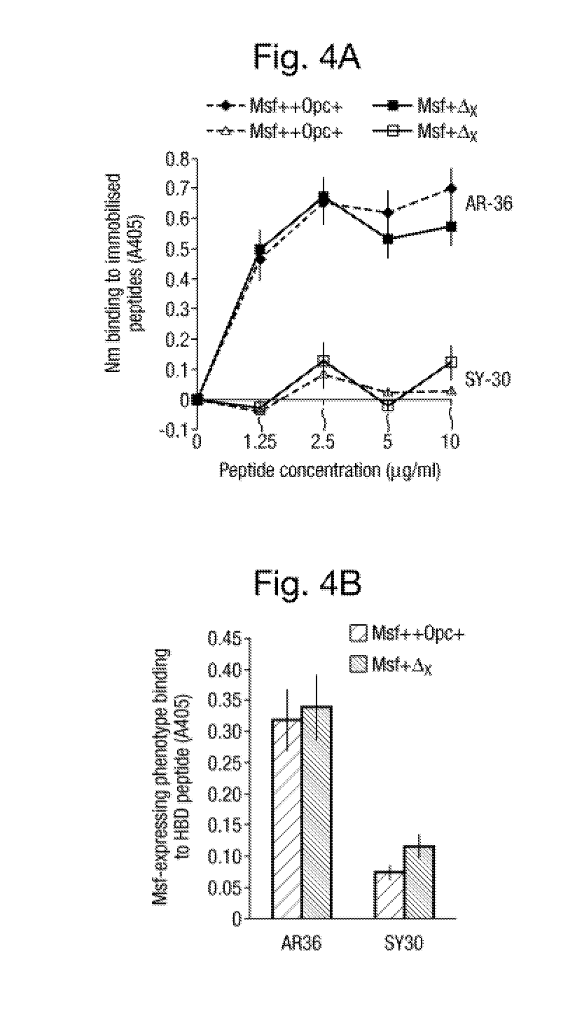

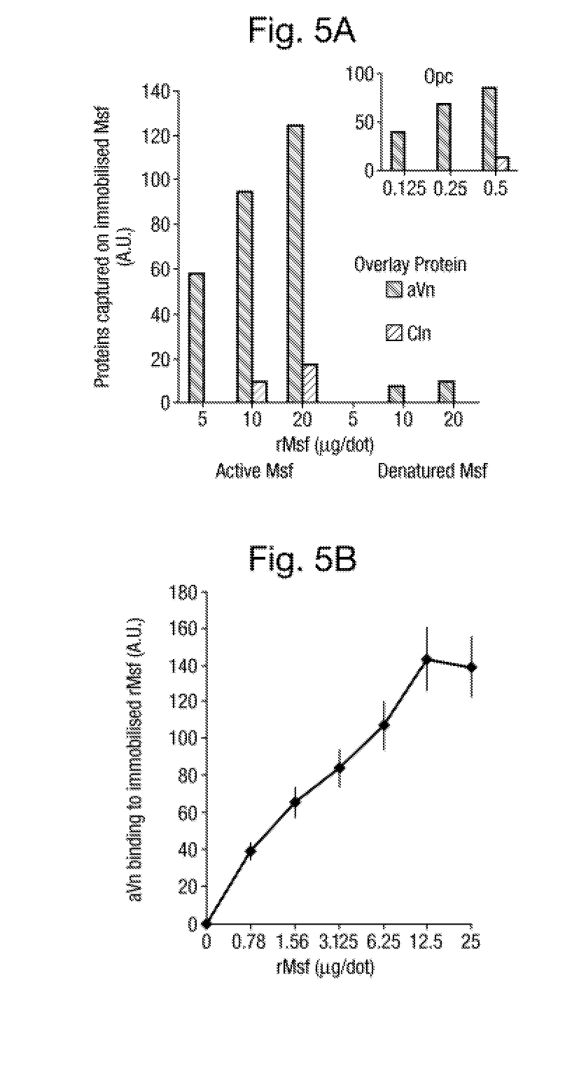

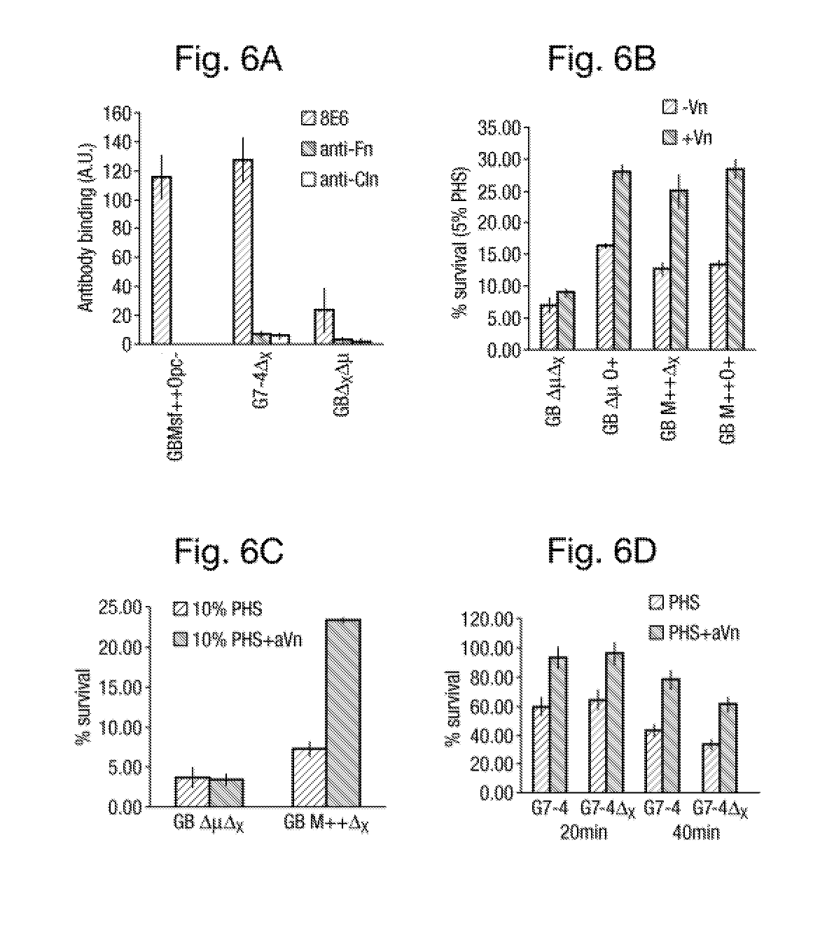

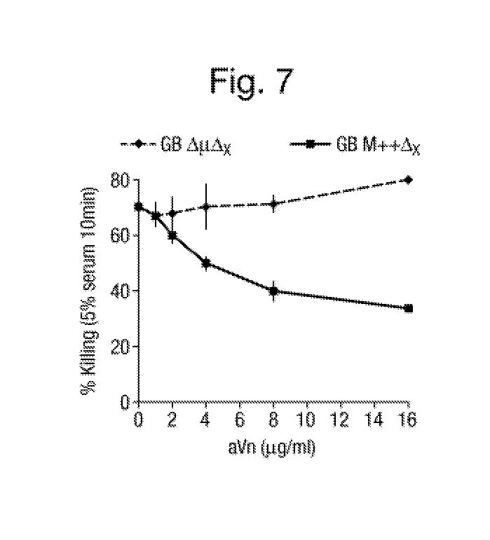

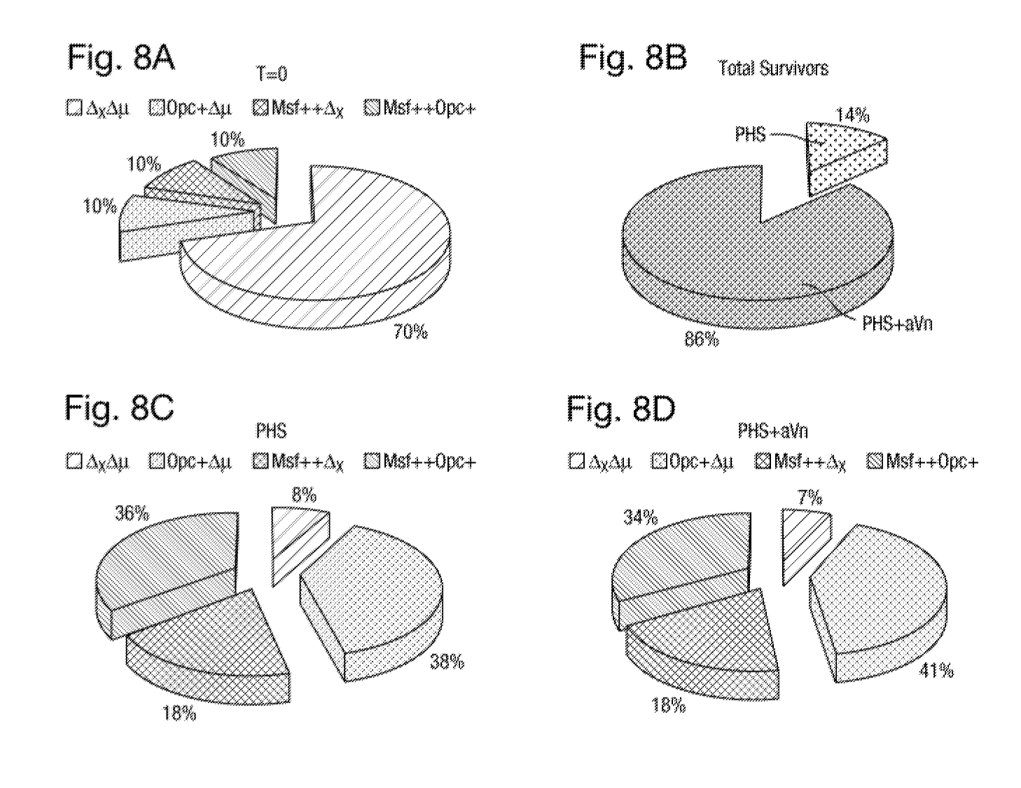

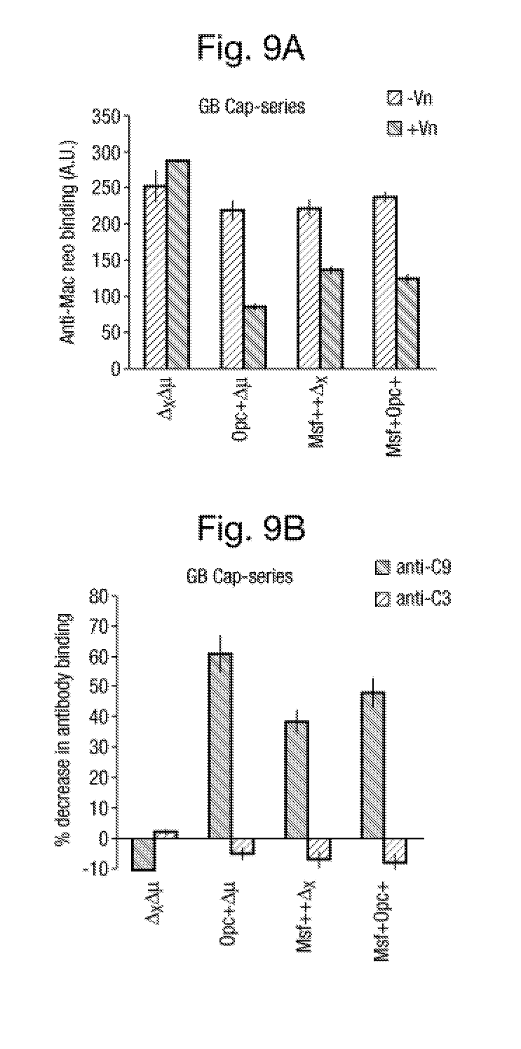

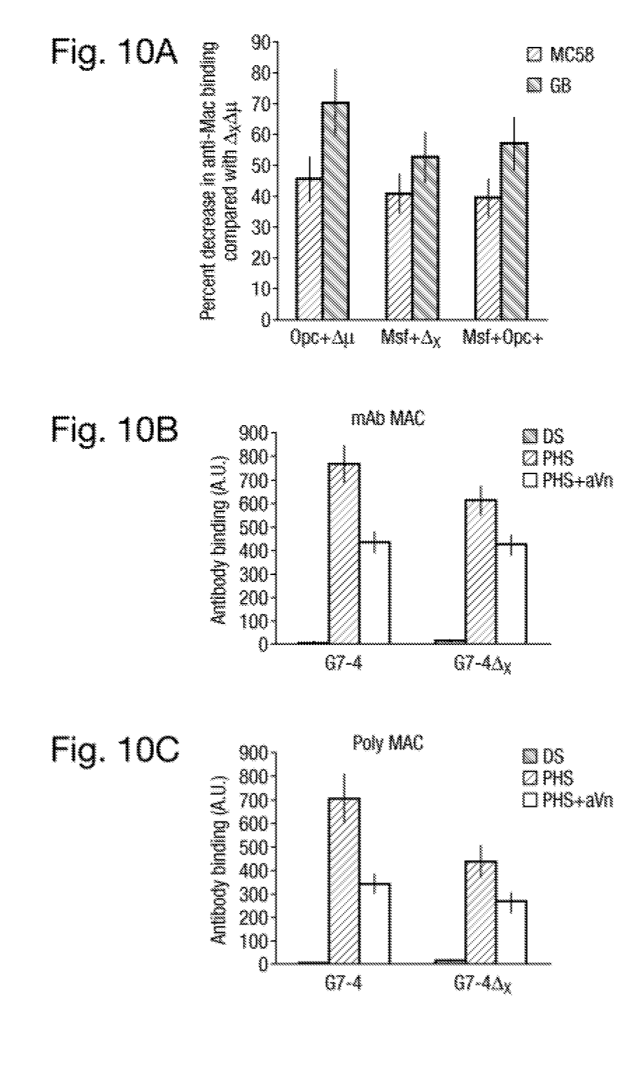

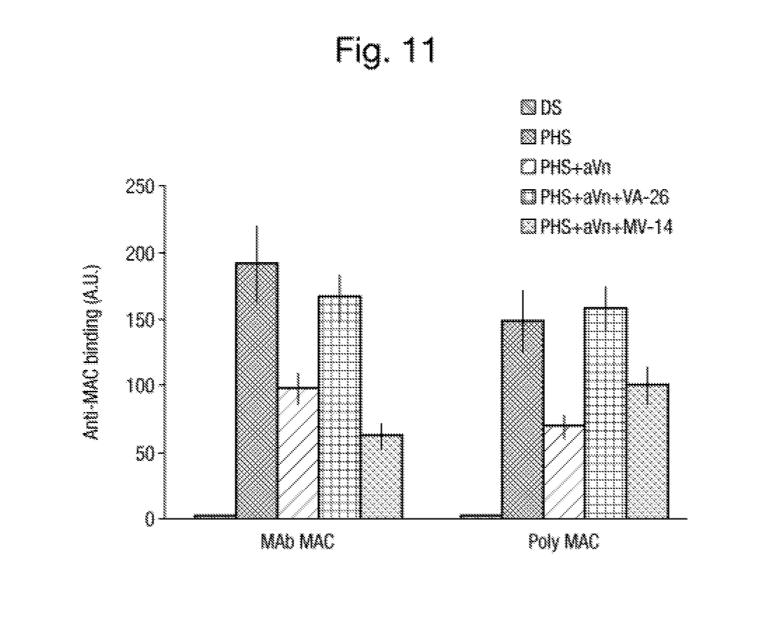

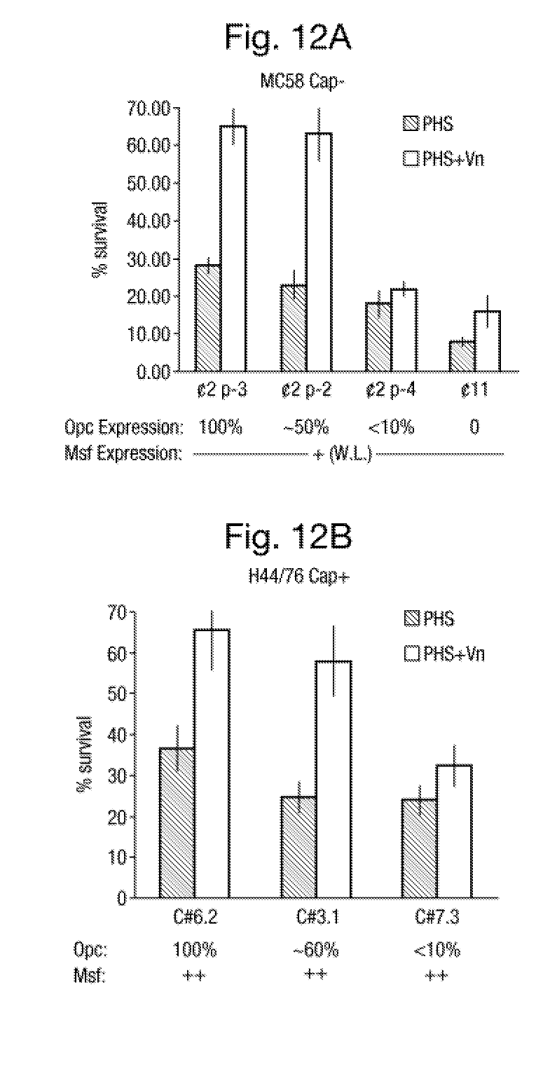

Using Nm derivatives which were proficient or deficient in Opc and Msf expression, we examined their ability to bind to aVn. In an ELISA, when whole cell suspensions of acapsulate H44/76 derivatives (Table 1) were overlaid on immobilised aVn, significantly increased binding of meningococci proficient in either of the adhesins over those with neither adhesin was immediately apparent (FIG. 1A). In addition, dual expressers bound aVn to the greatest extent, although the binding level of Msf+Opc+ was not always additive of the binding levels displayed by Msf+Opc− and Opc+Msf− phenotypes and depended on the experimental set up (FIGS. 1A and B).

Binding of Nm Isolates to Vitronectins from Animal Origins.

Vitronectin structure is highly conserved and the serum proteins derived from human, mouse, rabbit and bovine share extensive structural similarities. To study if the proteins from the animal origins are as well recognised as the human Vn, heparin-affinity purified preparations of the vitronectins were immobilised on ELISA plates and bacterial binding assessed. In each case, the Msf-expressing Nm bound to the immobilised activated vitronectins (FIG. 1C). These observations suggest that in animal models of infection or when animal sera are used in experimental procedures, the vitronectins of these distinct sources may be sequestered by certain meningococcal phenotypes as effectively as from human sera.

TABLE 1
Neisseria meningitidis derivatives used in the studies and their key
features.
A

| | | Nomenclature | | | |
|---|---|---|---|---|---|
| Strain | | used | Capsule | Msf | Opc |
| MC58 | | (58)Msf+ Opc+ | + | + | + |
| | | (58) Msf+ Δχ | + | + | − |
| | | (58) Opc+Δμ | + | − | + |
| | | (58) Δμ Δχ | + | − | − |
| H44/76 | Series #1 | (GB) Msf++ Opc+ | − | ++ | + |
| | Opc− variants | (GB) Msf++ Opc− | − | ++ | − |
| | | (GB) Opc+Δμ | − | − | + |
| | | (GB) Opc−Δμ | − | − | − |
| | Series #2 | (GB) Msf++ Opc+ | − | ++ | + |
| | Opc− mutants | (GB) Msf++ Δχ | − | ++ | − |
| | | (GB) Opc+Δμ | − | − | + |
| | | (GB) Δμ Δχ | − | − | − |

B

| Strain origin | | Nomenclature | | | |
|---|---|---|---|---|---|
| (original ID) | Msf origin | used | Capsule | Msf | Opc |
| MOI-240101 | endogenous | G7-1 | + | ++ | +/− |
| (B2472) | | | | | |
| MOI-240101 | endogenous | G7-2 | ++ | + | +/− |
| (B2473) | | | | | |
| H44/76 | MOI-240101 | G7-3 | − | ++ | +/− |
| (B2474) | | | | | |
| H44/76 | B16B6 | G7-4 | ++ | +++ | +/− |
| (B2458) | | | | | |

Opc mutants:

| | | | | |
|---|---|---|---|---|
| B2472 ΔOpc | G7-1 Δχ | + | ++ | − |
| B2473 ΔOpc | G7-2 Δχ | ++ | + | − |
| B2474 ΔOpc | G7-3 Δχ | − | ++ | − |
| B2458 ΔOpc | G7-4 Δχ | ++ | +++ | − |

‘Opc−’ refers to naturally arising Opc-deficient variants.
‘Opc+’ refers to naturally arising high Opc-expressing variants.
‘Δχ’ refers to Opc mutants.
‘Msf++’ refers to derivatives engineered for over-expression of Msf (GB series below).
‘Msf+’ refers to natural variants with moderate levels of Msf expression (<50% that in GB).
‘Δμ’ refers to Msf mutants.

### Characterisation of the Binding Region of Msf on Human Vitronectin

To assess which regions of the proteins might be targeted by the two proteins, several synthetic vitronectin peptides were used. Previously, Opc was shown to bind to tyrosine sulphated region of Vn. The use of the peptide spanning the residues V43-A68 (VA-26), its sulphated derivative VA-26S (sulphated at Y56 and Y59) and its phosphorylated peptide VA-26P (phosphorylated at T50 and T57) demonstrated specific inhibition of Opc binding to aVn by VA-26S but not VA-26 or VA-26P (Sa E Cunha, Griffiths et al. 2010). These peptides were used in the current studies to assess if any region of the peptides may be recognised by Msf.

In direct binding studies, significant binding of Msf-expressing derivatives to all three immobilised biotinylated peptides was seen (FIG. 2 A-C). In contrast, Opc expressing Msf− Nm only bound significantly to VA-26S as observed previously (Sa E Cunha, Griffiths et al. 2010). Specific binding of VA-26S to both Msf and Opc was shown in an ELISA with immobilised peptides VA-26S and two unrelated peptides SY-30 and GS-22 with no sequence similarity to Vn. No binding of either of the control peptides was seen (FIG. 2D).

To confirm the vitronectin region involved in binding of Msf vs. Opc to Vn, bacterial binding was assessed using either VA-26S peptide that should inhibit Msf and Opc binding (from above studies) and 8E6 mAb which is known to inhibit Opc binding (Sa E Cunha, Griffiths et al. 2010). Indeed, VA-26S and 8E6 abrogated Opc binding to aVn as expected, since direct binding of Opc to Vn occurs at the tyrosine sulphated region only (Sa E Cunha, Griffiths et al. 2010). However, only 30% reduction in Msf binding was observed with VA-26S and even less (10%) with the mAb 8E6. In the case of Msf+Opc+ phenotype, the peptide and 8E6 were both more effective than with Msf+Opc− derivative. Overall, the data suggest that i) Msf and Opc both bind to aVn in the phenotype expressing these proteins simultaneously, ii) that Msf binding region may be located partly within the V43-A68 region of human vitronectin and iii) that VA-26S peptide alone is not sufficient to abrogate Msf binding to aVn. Thus it is possible that Vn provides a fuller or more native epitope compared with VA-26S and/or the monomeric peptide cannot compete well with the multimeric activated Vn. In addition, other sites on aVn may also be directly targeted by Msf.

### The Vn Peptide VA-26 is Recognised by Msf Proteins of Distinct Strains.

The Msf proteins of strains MO1-240101 and B16B6 are structurally most dissimilar to H44/76 amongst the known Msf proteins (although their overall identity is >85%; see appendix 4). To assess their ability to bind to the synthetic VA-26 peptide, whole cell bacterial lysates from Nm derivatives over-expressing these proteins were used in an ELISA. The levels of binding to VA-26 correlated with the differences in the levels of Msf expressed rather than the small structural differences between the Msf proteins (FIG. 3).

### Msf has the Potential to Bind to the Heparin-Binding Domain of Vitronectin.

Several bacteria bind to the basic heparin-binding domain (HBD) of human Vn either directly or indirectly. We have shown that Opc can bind to aVn HBD by bridging via heparin (see Appendix 1 of the priority document). To investigate if direct or indirect interactions occur via Msf, we used a synthetic biotinylated peptide AR36 spanning the Vn residues A360-R395 at the C terminal region of Vn, the main heparin binding site, and examined the binding of acapsulate H44/76 isolates. In contrast to Opc, Msf bound to the HBD peptide directly (FIG. 4).

### Purified Msf Protein Binds to Activated Vitronectin but not to Clusterin.

To assess directly the interactions between Msf and aVn, recombinant His-tagged passenger domain of Msf (rMsf) was expressed in E. coli and its purification carried out under native conditions. Briefly, bacteria were harvested, re-suspended in Tris buffer, sonicated and cellular debris removed by centrifugation. The supernatant was then mixed with Ni-NTA agarose, packed into a column, washed, the rMsf eluted with imidazole-containing Tris-buffer and dialysed overnight. Such preparations of Msf were found in general to be functionally active and bound to activated human vitronectin as shown in FIG. 5. Some Msf preparations stored for several months had lost activity and such a preparation was used as a control.

### Serum Vitronectin Binding of Msf Isolates and Activated Vitronectin-Dependent Serum Resistance

To assess if Msf-expressing Nm bound to Vn from human serum, bacteria were incubated with serum, washed and the binding of aVn, fibronectin or clusterin assessed using specific antibodies against the human proteins. As shown in FIG. 6A, Msf expressing Nm bound significantly to aVn from serum but not to fibronectin or clusterin. Further, to assess if aVn increased serum resistance of Msf-expressing Nm, several phenotypes of H44/76 were exposed to serum and bacterial survival tested with 10% serum or 10% serum supplemented with 10 μg/ml aVn. (In most of the studies described here, in serum bactericidal assays (SBA), pooled normal human serum (PHS) from four individuals with no prior history of meningococcal disease was used; method for SBA is described in Appendix 2). Interestingly, either Msf or Opc expression led to increased serum resistance of Msf/Opc expressing phenotypes over those not expressing the protein, there was no additive effect in these experiments (FIG. 6). However, and importantly, Msf or Opc-expression was required for aVn-dependent increase in serum resistance (FIGS. 6B and C). To demonstrate the role of the variant B16B6 Msf in serum resistance and to assess its efficacy in capsulate phenotype, the H44-76 capsulate derivative expressing the heterologous B16B6 Msf was also used in a SBA. Activated Vn increased serum resistance of this phenotype also (FIG. 6D).

### Activated Vitronectin Concentration-Dependent Increase in Serum Resistance of Msf-Expressing Isolate.

Further demonstration of the association of aVn with increased serum resistance of Msf-expressing phenotype was obtained in an SBA carried out at a range of aVn concentrations. These experiments used Msf+ and Msf− H44/76 acapsulate derivatives. Due to their exquisite serum sensitivity, 5% NHS was used in these experiments. In such limited antibody situation, aVn halved the serum killing when present at 16 μg/ml (maximum tested, FIG. 7).

### Fittest Phenotype Study.

To illustrate the survival advantage of the Msf or Opc expressing phenotypes in normal human serum, strain H44/76 derivatives were mixed such that the initial population comprised 70% non-expressers to 30% of single or dual expressers in equal proportions (FIG. 8A). After exposure to 10% PHS (with or without added aVn) for 10 min, the survivors were plated and colony forming units (cfu) determined. First, the total survivors in PHS with added aVn exceeded those in PHS alone by 6-7-fold. Second, within 10 minutes, the phenotypes expressing one or both of the vitronectin-binding proteins predominated such that together, they constituted >90% of the total population in either PHS or in aVn-supplemented PHS (FIGS. 8C and D). Examination of the population structure revealed that the ratio of the Msf/Opc expressing phenotypes Opc+: Msf+: Opc/Msf+ increased from 1:1:1 to approximately 4:1.8:3.5. As the survival of each phenotype will also depend on the antibody composition of the serum which may contain blocking antibodies, it is notable that using sera from randomly selected donors, the exquisite efficacy of the two proteins in increasing serum resistance of meningococci could be demonstrated. The similarity in the final profile of surviving populations (FIGS. 8C and D) also reflects the similarity of the mechanisms of survival in unsupplemented PHS and aVn-supplemented PHS.

The Terminal Complement C5b-9 Deposition but not C3 Deposition is Affected by Msf/Opc-Interactions with Activated Vn.

Activated Vn associates with its physiological ligands including C5b-7 and C5b-9 terminal complement complexes. Its binding to C9 component during the formation of C5b-9 (MAC) inhibits the polymerisation of C9, prevents the formation of C9 lytic pore into the target membrane and thus prevents cell lysis. During the polymerisation of C9, neoantigens on C9 are revealed. A monoclonal antibody against a C9 neoantigen (this experiment) as well as a polyclonal antibody recognising C9 in MAC complex (data shown in FIG. 10) were used to assess the level of C9 associated with bacterial membranes following aVn addition to PHS. To assess if the observed decrease in serum bactericidal activity in aVn-supplemented PHS is primarily due to the inhibition of the terminal stages of the complement pathway, the effect of added Vn on the deposition of C3 on bacteria was also assessed using a polyclonal anti-C3 antibody. Data shown in FIG. 9 demonstrate the specific control of MAC deposition with added aVn.

Binding of Msf/Opc Expressing Capsulate Phenotypes to aVn Also Leads to the Inhibition of MAC Insertion into Bacterial Membranes.

In further studies, MAC deposition on acapsulate and capsulate derivatives with different Msf proteins were determined using capsulate strain MC58 and acapsulate H44/76 expressing their respective Msf proteins (FIG. 10A) and capsulate H44/76 derivative expressing the heterologous B16B6 Msf (FIG. 10B).

First, comparison of the distinct MC58 and H44/76 phenotypes in a SBA (using aVn-supplemented PHS) revealed much lower MAC deposition when Opc and/or Msf were expressed compared to Msf-Opc− derivatives (FIG. 10A).

In further experiments, C9 associated with capsulate G7-4 derivatives was examined, again revealing decreased association of C9 in aVn-supplemented PHS.

### Inhibition of Meningococcal-Vitronectin Interactions: Synthetic Vitronectin Peptides Restore MAC Deposition.

As is apparent from the studies presented in FIG. 2, synthetic peptides spanning the vitronectin residues V43-A68 (e.g. VA-26) significantly inhibit Msf interactions with aVn. To assess the efficacy of such peptides in restoring MAC deposition on bacteria, VA-26 was used in a MAC deposition assay which also included a control peptide, MV-14 (FIG. 11). Consistent with its role in inhibiting aVn interactions with Msf, VA-26 but not the control peptide MV-14 restored MAC deposition.

Binding of Msf to Vn in the SMB/Linker Region of aVn does not Lead to Targeting of Human Cellular Integrins.

We have shown that vitronectin can form a bridge between Opc on Nm and endothelial cell integrins to increase bacterial cell adhesion and invasion. However, in our current studies, we have not observed similar cell targeting by Msf-expressing Nm. There is evidence that in Msf/Opc dual expressing phenotypes, Msf may inhibit Opc-mediated cellular interactions (data not shown). It appears that the region of Vn targeted by Msf is upstream of the tyrosine sulphated Opc binding site and may overlap the ‘RGD’ cell binding region of Vn. This could explain the lack of endothelial cell binding of Msf-expressing phenotypes. In addition, due to the close proximity of their binding sites on vitronectin, Opc-dependent cell adhesion could also be hampered by Msf.

### FINAL COMMENTS

Complement resistance is an essential property for successful colonisation of mucosal and submucosal environments and Neisseria meningitidis, a frequent coloniser of the human nasopharynx, has evolved several strategies for this purpose. These include elaboration of surface sialic acids common to meningococci which assist in antibody and complement evasion through complex mechanisms, some of which involve molecular mimicry. Another common strategy entails sequestration of the host complement evasion proteins via a number of outer membrane proteins including the opacity protein Opc, the protein known as GNA1870 (LP2086) or factor H binding protein (fHbp), Neisserial surface protein A (NspA) and the porin protein PorA. These proteins have been shown to bind to distinct host molecules such as vitronectin (Opc), factor H (fHbp and NspA) and C4bP (PorA), although the latter binding did not occur at physiological salt concentrations. Vn, fH and C4BP control different stages of complement pathways: fH down modulates the alternative pathway (AP) by retarding the formation of, and dissociating preformed, C3 convertase; C4BP has a similar effect on the classical pathway (CP) C4 convertase; whereas vitronectin by preventing the terminal C5b-9 membrane attack complex (MAC) insertion into cell membranes, exerts its effect at the terminal stages of complement deposition at which all complement pathways converge. Thus binding to Vn by meningococci could afford considerable advantage to bacteria even in an immune host. We have shown that Msf, like Opc, is a vitronectin binding protein with the ability to resist complement attack. In doing so, this study has identified a novel and an important mechanism of overall complement resistance.

Msf may be upregulated on host cell contact (Sjolinder, Eriksson et al. 2008) (Hartman, Virji and Heyderman, IPNC 2002). In addition, several studies have shown that recombinant Msf is immunogenic and elicits bactericidal antibodies, and that convalescent sera contain anti-Msf antibodies. As an immunogenic protein which may have special functions during infection, it is important to establish the potential of the protein in colonisation and disease. Antibody and complement play critical roles in defence against meningococci, upregulation of the Msf protein during infection and its potential to resist the actions of all complement pathways makes it an important target molecule for in depth investigation.

Identification of its host targets, such as vitronectin and its features required for these interactions, will be beneficial from the following points of view. Retaining structural features of vaccine candidates such as Msf, that may elicit blocking antibodies, could be particularly beneficial as these antibodies, by inhibiting vitronectin sequestration, could increase the efficacy of all antibodies against bacteria and could themselves serve as bactericidal antibodies. Further, as Opc at low/intermediate levels could assist in serum resistance as described in Appendix 3, Opc and Msf together in vaccine preparations may serve to control the spread of a wide range of virulent meningococcal strains.

### REFERENCES

Hogasen, K., T. E. Mollnes, et al. (1994). “Low levels of vitronectin and clusterin in acute meningococcal disease are closely associated with formation of the terminal-complement complex and the vitronectin-thrombin-antithrombin complex.” Infect Immun 62(11): 4874-80.
Peak, I. R., Y. Srikhanta, et al. (2000). “Identification and characterisation of a novel conserved outer membrane protein from Neisseria meningitidis.” FEMS Immunol Med Microbiol 28(4): 329-34.
Sa E Cunha, C., N. J. Griffiths, et al. (2010). “Neisseria meningitidis Opc invasin binds to the sulphated tyrosines of activated vitronectin to attach to and invade human brain endothelial cells.” PLoS Pathog 6(5): e1000911.
Sjolinder, H., J. Eriksson, et al. (2008). “Meningococcal outer membrane protein NhhA is essential for colonization and disease by preventing phagocytosis and complement attack.” Infect Immun 76(11): 5412-20.
Virji, M., K. Makepeace, et al. (1994). “Distinct Mechanisms of Interactions of Opc-Expressing Meningococci at Apical and Basolateral Surfaces of Human Endothelial-Cells—the Role of Integrins in Apical Interactions.” Molecular Microbiology 14(1): 173-184.
Virji, M., K. Makepeace, et al. (1995). “Opc- and pilus-dependent interactions of meningococci with human endothelial cells: molecular mechanisms and modulation by surface polysaccharides.” Mol. Microbiol. 18(4): 741-754.
Hartman G., Virji M. and Heyderman R.: Differential expression of NMB1856 (crgA), and the autotransporter homologues NMB1985 and NMB0992 by Neisseria meningitidis during early interactions with human epithelial cells. Thirteenth International Pathogenic Neisseria Conference. Norwegian Institute of Public Health, Oslo, Norway Norway; Sep. 1-6, 2002.

### Note: NMB0992=NhhA=Msf.

### APPENDICES

### 2. Serum Bactericidal Assay (SBA)

### Endogenous Antibody and Complement

Bacterial suspensions were prepared in Dulbecco's phosphate buffered saline, enumerated and the required dilution of freshly thawed human serum immediately added to 10³ bacteria in 100 μl volume. After incubation at 37° in a CO₂ incubator (usually for 10 min for acapsulate and 30 min for capsulate meningococci), dilutions of bacterial suspensions were plated on agar to determine the numbers of surviving bacteria. Percent killing was then calculated by comparison with the numbers of bacteria exposed in a similar manner to decomplemented serum (56° C., 30 min).

### 3. Further Studies on the Effect of Varying Opc Levels on Serum Resistance of Capsulate and Acapsulate Msf-Expressing Phenotypes

Opc levels can vary in vivo and it is suggested that levels of the protein in mucosal isolates are high whereas in blood isolates they tend to be low. To assess the effect of Opc levels on serum resistance, two series of derivatives were used to represent in vivo colonisation and disease phenotypes. MC58 acapsulate derivatives of ⊂/ 2 series express levels of Msf, which are approximately 30% of those expressed in the over-expressing H44/76 derivatives. Notably H44/76 and MC58 Msf are identical and Opc proteins are largely invariant in all meningococci (identities are shown in appendix 4). In both strains, Opc derivatives with a range of expression levels were selected as shown in the figure below. In MC58 with low/moderate Msf expression, Msf alone increases serum resistance (15% greater survival in aVn-supplemented PHS—see Opc mutant ⊂/11, FIG. 12A). In both series of isolates, increasing Opc levels aid in serum resistance to a moderate extent in PHS and much more dramatically in aVn-supplemented PHS. It is also apparent that both in acapsulate and capsulate background, less than maximum levels of Opc are sufficient to increase serum to resistance considerably (FIG. 12).

### 4. Tables Showing Opc and Msf Relatedness of Various Meningococcal Strains

### Opc Relatedness of Neisseria meningitidis Strains

| Percent Identity | | | | | | | | | | | |
|---|---|---|---|---|---|---|---|---|---|---|---|
| | 1 | 2 | 3 | 4 | 5 | 6 | 7 | 8 | 9 | | |
| 1 | | 88.8 [symbol] | 100.0 | 89.5 [symbol] | 87.8 [symbol] | 98.3 [symbol] | 99.6 [symbol] | 38.4 [symbol] | 92.3 [symbol] | 1 | Z2431 Opo.pro [symbol] |
| 2 | 0.4 | | 88.8 [symbol] | 100.0 | 97.4 [symbol] | 97.9 [symbol] | 99.1 [symbol] | 97.6 [symbol] | 92.6 [symbol] | 2 | ND60 Opc.pro [symbol] |
| 3 | 0.0 | 0.4 | | 69.0 [symbol] | 97.8 [symbol] | 88.3 [symbol] | 99.0 [symbol] | 90.4 [symbol] | 99.0 [symbol] | 3 | Z3470 Opo.cro [symbol] |
| 4 | 0.4 | 0.0 | 0.4 | | 97.4 [symbol] | 97.9 [symbol] | 98.1 [symbol] | 98.8 [symbol] | 92.6 [symbol] | 4 | NC131 Opo.pro [symbol] |
| 5 | 2.2 | 2.0 [symbol] | 2.2 | 2.6 | | 85.7 [symbol] | 98.5 [symbol] | 98.1 [symbol] | 97.4 [symbol] | 5 | NC138 Opo.pro [symbol] |
| 6 | 1.7 [symbol] | 2.1 | 1.7 | 2.1 [symbol] | 1.3 | | 57.9 [symbol] | 80.0 [symbol] | 97.5 [symbol] | 6 | NC118 Opc.pro [symbol] |
| 7 | 0.4 [symbol] | 0.9 [symbol] | 0.4 [symbol] | 0.9 [symbol] | 1.7 [symbol] | 2.3 [symbol] | | 97.9 [symbol] | 99.1 [symbol] | 7 | NC122 Opc.oro [symbol] |
| 8 | 2.7 [symbol] | 2.5 [symbol] | 1.7 [symbol] | 1.3 | 4.0 | 3.4 [symbol] | 3.3 [symbol] | | 88.4 [symbol] | 8 | 8235 Opo.pro [symbol] |
| 9 | 0.3 [symbol] | 0.4 | 0.7 [symbol] | 0.4 | 2.6 [symbol] | 2.0 [symbol] | 0.3 [symbol] | 3.7 | | 9 | 515P1 10 Opo.pro |
| | 1 | 2 | 3 | 4 | 5 | 6 | 7 | 8 | 9 | | |

[symbol]

indicates data missing or illegible when filed

### Msf Relatedness of Neisseria Meningitidis Strains

### Comparison of MC58 with 1-144/76 and the Least Related Msf of Z2491, MO1-240101 and B16B6

| Parent Identity | | | | | | | |
|---|---|---|---|---|---|---|---|
| | 1 | 2 | 3 | 4 | 5 | | |
| 1 | | 100.0 | 98.9 [symbol] | 82.7 [symbol] | 85.0 [symbol] | 1 | Hef-MC58.pro [symbol] |
| 2 | 0.0 [symbol] | | 88.8 [symbol] | 88.7 [symbol] | 86.0 [symbol] | 2 | H44-70-Hsfpro [symbol] |
| 3 | 14.4 | 14.4 | | 92.0 [symbol] | 91.1 [symbol] | 3 | 22481 Hs-pro [symbol] |
| 4 | 12.2 | 12.2 | 8.5 [symbol] | | 84.7 [symbol] | 4 | NDI-24C0C0.pro [symbol] |
| 5 | 15.5 | 15.5 | 9.5 | 5.5 [symbol] | | 5 | 515E0.pro [symbol] |
| | 1 | 2 | 3 | 4 | 5 | | |

[symbol]

indicates data missing or illegible when filed

[symbol]

1. An immunogenic composition comprising neisserial Hsf and Opc antigens.

2. The immunogenic composition of claim 1 further comprising neisserial FHbp antigen.

3. (canceled)

4. The immunogenic composition of claim 1, wherein the Hsf antigen selected from the group of:
a) a polypeptide comprising the amino acid sequence of SEQ ID NO: 2;
b) a polypeptide comprising an amino acid sequence which has at least 80% sequence identity with SEQ ID NO: 2;
c) a polypeptide comprising an immunogenic fragment of at least 10 contiguous amino acids of SEQ ID NO:2;
d) a peptide or polypeptide capable of eliciting antibodies which can bind a polypeptide of SEQ ID NO: 2 and/or can bind a N. meningitidis bacterium expressing a polypeptide of SEQ ID NO: 2 within its outer membrane; and,
e) a peptide or polypeptide capable of eliciting antibodies which can inhibit the binding of human vitronectin to a polypeptide of SEQ ID NO: 2 and/or can inhibit the binding of human vitronectin to a N. meningitidis bacterium expressing a polypeptide of SEQ ID NO: 2 within its outer membrane.

5.-7. (canceled)

8. The immunogenic composition of claim 1, wherein the Hsf antigen is present in the immunogenic composition at a dose sufficient to elicit antibodies in a human host which can inhibit the binding of human vitronectin to Hsf and/or can inhibit the binding of human vitronectin to a N. meningitidis bacterium expressing Hsf within its outer membrane.

9.-20. (canceled)

21. The immunogenic composition of claim 1, wherein the Opc antigen is selected from the group of:
a) a polypeptide comprising the amino acid sequence of SEQ ID NO: 4;
b) a polypeptide comprising an amino acid sequence which has at least 80% sequence identity with SEQ ID NO: 4;
c) a polypeptide comprising an immunogenic fragment of at least 10 contiguous amino acids of SEQ ID NO:4;
d) a peptide or polypeptide capable of eliciting antibodies which can bind a polypeptide of SEQ ID NO: 4 and/or can bind a N. meningitidis bacterium expressing a polypeptide of SEQ ID NO: 4 within its outer membrane; and
e) a peptide or polypeptide capable of eliciting antibodies which can inhibit the binding of human vitronectin to a polypeptide of SEQ ID NO: 4 and/or can inhibit the binding of human vitronectin to a N. meningitidis bacterium expressing a polypeptide of SEQ ID NO: 4 within its outer membrane.

22.-24. (canceled)

25. The immunogenic composition of claim 1, wherein the Opc antigen is present in the immunogenic composition at a dose sufficient to elicit antibodies in a human host which can inhibit the binding of human vitronectin to Opc and/or can inhibit the binding of human vitronectin to a N. meningitidis bacterium expressing Opc within its outer membrane.

26.-38. (canceled)

39. The immunogenic composition of claim 2, wherein the FHbp antigen is from family A or family B.

40. The immunogenic composition of claim 39, wherein the FHbp family A antigen is selected from the group of:
a) a polypeptide comprising the amino acid sequence of SEQ ID NO: 5;
b) a polypeptide comprising an amino acid sequence which has at least 70% sequence identity with SEQ ID NO: 5;
c) a polypeptide comprising an immunogenic fragment of at least 10 contiguous amino acids of SEQ ID NO:5;
d) a peptide or polypeptide capable of eliciting antibodies which can bind a polypeptide of SEQ ID NO: 5 and/or can bind a N. meningitidis bacterium expressing a polypeptide of SEQ ID NO: 5 within its outer membrane; and,
e) a peptide or polypeptide capable of eliciting antibodies which can inhibit the binding of human factor H to a polypeptide of SEQ ID NO: 5 and/or can inhibit the binding of human factor H to a N. meningitidis bacterium expressing a polypeptide of SEQ ID NO: 5 within its outer membrane.

41.-43. (canceled)

44. The immunogenic composition of claim 39, wherein the FHbp family A antigen is present in the immunogenic composition at a dose sufficient to elicit antibodies in a human host which can inhibit the binding of human factor H to FHbp family A and/or can inhibit the binding of human factor H to a N. meningitidis bacterium expressing FHbp family A within its outer membrane.

45. The immunogenic composition of claim 39, wherein the FHbp family B antigen is selected from the group of:
a) a polypeptide comprising the amino acid sequence of SEQ ID NO: 6;
b) a polypeptide comprising an amino acid sequence which has at least 70, 80, 85, 90, 95, or 99% sequence identity with SEQ ID NO:
c) a polypeptide comprising an immunogenic fragment of at least 10 contiguous amino acids of SEQ ID NO:6;
d) a peptide or polypeptide capable of eliciting antibodies which can bind a polypeptide of SEQ ID NO: 6 and/or can bind a N. meningitidis bacterium expressing a polypeptide of SEQ ID NO: 6 within its outer membrane; and,
e) a peptide or polypeptide capable of eliciting antibodies which can inhibit the binding of factor H to a polypeptide of SEQ ID NO: 6 and/or can inhibit the binding of human factor H to a N. meningitidis bacterium expressing a polypeptide of SEQ ID NO: 6 within its outer membrane.

46.-48. (canceled)

49. The immunogenic composition of claim 39, wherein the FHbp family B antigen is present in the immunogenic composition at a dose sufficient to elicit antibodies in a human host which can inhibit the binding of human factor H to FHbp family B and/or can inhibit the binding of human factor H to a N. meningitidis bacterium expressing FHbp family B within its outer membrane.

50.-60. (canceled)

61. The immunogenic composition of claim 1, further comprising at least one N. meningitidis NspA and/or PilC antigen.

62. The immunogenic composition of claim 1, further comprising one or more bacterial capsular polysaccharides or oligosaccharides.

63. The immunogenic composition of claim 62 wherein the capsular polysaccharides or oligosaccharides are derived from bacteria selected from the group consisting of: Neisseria meningitidis serogroup A, C, Y and W-135, Haemophilus influenzae b, Streptococcus pneumoniae, Group A Streptococci, Group B Streptococci, Staphylococcus aureus and Staphylococcus epidermidis.

64.-67. (canceled)

68. A vaccine comprising the immunogenic composition of claim 1, and a pharmaceutically acceptable carrier.

69. A method for treatment or prevention of Neisserial disease comprising administering a protective dose of the vaccine of claims 68 to a host in need thereof.

70. The method of claim 69 in which Neisseria meningitidis and/or Neisseria gonorrhoeae infection is prevented or treated.

71.-79. (canceled)

80. A method of making the immunogenic composition of claim 1, comprising a step of mixing together the Hsf and Opc antigens.

81.-82. (canceled)

83. A pharmaceutical composition comprising antibodies specific for the Hsf and Opc antigens of the immunogenic composition of claim 1.

84. (canceled)


SEQUENCE LISTING (17 sequences)

SEQ ID NO: 1 - DNA, 1776 bp, Neisseria meningitidis
atgaacaaaatataccgcatcatttggaatagtgccctcaatgcctgggtcgtcgtatccgagctcacacgcaaccacaccaaacgcgcctccgcaaccgtgaagaccgccgtattggcgacactgttgtttgcaacggttcaggcaagtgctaacaatgaagagcaagaagaagatttatatttagaccccgtacaacgcactgttgccgtgttgatagtcaattccgataaagaaggcacgggagaaaaagaaaaagtagaagaaaattcagattgggcagtatatttcaacgagaaaggagtactaacagccagagaaatcaccctcaaagccggcgacaacctgaaaatcaaacaaaacggcacaaacttcacctactcgctgaaaaaagacctcacagatctgaccagtgttggaactgaaaaattatcgtttagcgcaaacggcaataaagtcaacatcacaagcgacaccaaaggcttgaattttgcgaaagaaacggctgggacgaacggcgacaccacggttcatctgaacggtattggttcgactttgaccgatacgctgctgaataccggagcgaccacaaacgtaaccaacgacaacgttaccgatgacgagaaaaaacgtgcggcaagcgttaaagacgtattaaacgctggctggaacattaaaggcgttaaacccggtacaacagcttccgataacgttgatttcgtccgcacttacgacacagtcgagttcttgagcgcagatacgaaaacaacgactgttaatgtggaaagcaaagacaacggcaagaaaaccgaagttaaaatcggtgcgaagacttctgttattaaagaaaaagacggtaagttggttactggtaaagacaaaggcgagaatggttcttctacagacgaaggcgaaggcttagtgactgcaaaagaagtgattgatgcagtaaacaaggctggttggagaatgaaaacaacaaccgctaatggtcaaacaggtcaagctgacaagtttgaaaccgttacatcaggcacaaatgtaacctttgctagtggtaaaggtacaactgcgactgtaagtaaagatgatcaaggcaacatcactgttatgtatgatgtaaatgtcggcgatgccctaaacgtcaatcagctgcaaaacagcggttggaatttggattccaaagcggttgcaggttcttcgggcaaagtcatcagcggcaatgtttcgccgagcaagggaaagatggatgaaaccgtcaacattaatgccggcaacaacatcgagattacccgcaacggtaaaaatatcgacatcgccacttcgatgaccccgcagttttccagcgtttcgctcggcgcgggggcggatgcgcccactttgagcgtggatggggacgcattgaatgtcggcagcaagaaggacaacaaacccgtccgcattaccaatgtcgccccgggcgttaaagagggggatgttacaaacgtcgcacaacttaaaggcgtggcgcaaaacttgaacaaccgcatcgacaatgtggacggcaacgcgcgtgcgggcatcgcccaagcgattgcaaccgcaggtctggttcaggcgtatttgcccggcaagagtatgatggcgatcggcggcggcacttatcgcggcgaagccggttacgccatcggctactccagtatttccgacggcggaaattggattatcaaaggcacggcttccggcaattcgcgcggccatttcggtgcttccgcatctgtcggttatcagtggtaa

SEQ ID NO: 2 - protein, 591 aa, Neisseria meningitidis
MNKIYRIIWNSALNAWVVVSELTRNHTKRASATVKTAVLATLLFATVQASANNEEQEEDLYLDPVQRTVAVLIVNSDKEGTGEKEKVEENSDWAVYFNEKGVLTAREITLKAGDNLKIKQNGTNFTYSLKKDLTDLTSVGTEKLSFSANGNKVNITSDTKGLNFAKETAGTNGDTTVHLNGIGSTLTDTLLNTGATTNVTNDNVTDDEKKRAASVKDVLNAGWNIKGVKPGTTASDNVDFVRTYDTVEFLSADTKTTTVNVESKDNGKKTEVKIGAKTSVIKEKDGKLVTGKDKGENGSSTDEGEGLVTAKEVIDAVNKAGWRMKTTTANGQTGQADKFETVTSGTNVTFASGKGTTATVSKDDQGNITVMYDVNVGDALNVNQLQNSGWNLDSKAVAGSSGKVISGNVSPSKGKMDETVNINAGNNIEITRNGKNIDIATSMTPQFSSVSLGAGADAPTLSVDGDALNVGSKKDNKPVRITNVAPGVKEGDVTNVAQLKGVAQNLNNRIDNVDGNARAGIAQAIATAGLVQAYLPGKSMMAIGGGTYRGEAGYAIGYSSISDGGNWIIKGTASGNSRGHFGASASVGYQW

SEQ ID NO: 3 - DNA, 819 bp, Neisseria meningitidis
atgaaaaaaacagtttttacatgtgccatgattgccctgaccggtactgccgccgctgcacaagagcttcaaaccgctaatgagtttaccgtccacaccgacctctcttccatttcttcaactcgtgctttcctgaaagaaaaacacaaagctgccaaacacatcagcgtgcgtgctgatattccttttgatgccaaccaaggcatccgcttggaagccggtttcgggcgcagcaaaaaaaatattattaatttggaaacagatgagaacaagctgggtaagactaaaaatgtaaaactgcccaccggcgttcctgaaaaccgtatcgatctttacacaggctacacctacacccaaacgttaagtgattctttaaatttccgtgtgggtgccggcttgggttttgaatcttcaaaagacagcattaaaaccaccaagcatacgcttcacagcagccgtcagtcgtggttagccaaagttcacgcggatttgctttcccaactgggtaacggctggtatatcaacccttggtctgaagtgaaatttgacctcaattcccgctataaattaaacaccggcgttaccaatctcaaaaaagacatcaatcaaaaaaccaacggctggggctttggattgggtgcaaatattggtaaaaaactgggcgaatccgccagcatcgaggcggggccgttctacaaacaacgcacttacaaagaatccggcgagtttagtgtaacaaccaagagtggcgacgtatcgctcaccatcccgaaaaccagtattcgtgaatacggcttgcgcgtcggcataaaattctga

SEQ ID NO: 4 - protein, 272 aa, Neisseria meningitidis
MKKTVFTCAMIALTGTAAAAQELQTANEFTVHTDLSSISSTRAFLKEKHKAAKHISVRADIPFDANQGIRLEAGFGRSKKNIINLETDENKLGKTKNVKLPTGVPENRIDLYTGYTYTQTLSDSLNFRVGAGLGFESSKDSIKTTKHTLHSSRQSWLAKVHADLLSQLGNGWYINPWSEVKFDLNSRYKLNTGVTNLKKDINQKTNGWGFGLGANIGKKLGESASIEAGPFYKQRTYKESGEFSVTTKSGDVSLTIPKTSIREYGLRVGIKF

SEQ ID NO: 5 - protein, 254 aa, Neisseria meningitidis
CSSGGGGVAADIGARLADALTAPLDHKDKSLQSLTLDQSVRKNEKLKLAAQGAEKTYGNGDSLNTGKLKNDKVSRFDFIRQIEVDGQLITLESGEFQIYKQDHSAVVALQIEKINNPDKIDSLINQRSFLVSGLGGEHTAFNQLPDGKAEYHGKAFSSDDAGGKLTYTIDFAAKQGHGKIEHLKTPEQNVELAAAELKADEKSHAVILGDTRYGSEEKGTYHLALFGDRAQEIAGSATVKIGEKVHEIGIAGKQ

SEQ ID NO: 6 - protein, 255 aa, Neisseria meningitidis
CSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 7 - protein, 23 aa, Neisseria meningitidis
  VARIANT (8)..(8): “X” is I/T
KINNPDKXDSLINXRSFLVSGLG

SEQ ID NO: 8 - protein, 23 aa, Neisseria meningitidis
  VARIANT (2)..(2): X is V/I/E
QXQDXEXSXXMVAKRXFXIGDIX

SEQ ID NO: 9 - protein, 5 aa, Neisseria meningitidis
  VARIANT (2)..(2): “X” is G/R
IXEKV

SEQ ID NO: 10 - protein, 5 aa, Neisseria meningitidis
  VARIANT (2)..(2): “X” is G/R
TXNGI

SEQ ID NO: 11 - protein, 30 aa, Artificial Sequence
  Biotinylated peptide SY-30
SGRGKGGKGLGKGGAKRHRKVLRDNIQGIY

SEQ ID NO: 12 - protein, 22 aa, Artificial Sequence
  Biotinylated peptide GS-22
GSNENQKTVKAESVPNMSFDQS

SEQ ID NO: 13 - protein, 36 aa, Artificial Sequence
  Biotinylated peptide AR36
APRPSLAKKQRFRHRNRKGYRSQRGHSRGRNQNSRR

SEQ ID NO: 14 - protein, 14 aa, Artificial Sequence
  Biotinylated peptide MV-14
MDFPVDTTEGPQRV

SEQ ID NO: 15 - protein, 26 aa, Artificial Sequence
  Biotinylated peptide VA-26
VTRGDVFTMPEDEYTVYDDGEEKNNA

SEQ ID NO: 16 - protein, 26 aa, Artificial Sequence
  Biotinylated peptide VA-26S
VTRGDVFTMPEDESTVSDDGEEKNNA

SEQ ID NO: 17 - protein, 26 aa, Artificial Sequence [sequence omitted]